SEQUENCE LISTING (22 sequences)

SEQ ID NO: 1 - protein, 5 aa, Artificial Sequence
  Synthetic Polypeptides
SHGIS

SEQ ID NO: 2 - protein, 17 aa, Artificial Sequence
  Synthetic Polypeptides
WISPYSGNTNYAQKLQG

SEQ ID NO: 3 - protein, 11 aa, Artificial Sequence
  Synthetic Polypeptides
VGSGPYYYMDV

SEQ ID NO: 4 - protein, 14 aa, Artificial Sequence
  Synthetic Polypeptides
TGTSSSVGDSIYVS

SEQ ID NO: 5 - protein, 7 aa, Artificial Sequence
  Synthetic Polypeptides
DVTKRPS

SEQ ID NO: 6 - protein, 9 aa, Artificial Sequence
  Synthetic Polypeptides
CSYAGTDTL

SEQ ID NO: 7 - protein, 9 aa, Artificial Sequence
  Synthetic Polypeptides
YSYAGTDTL

SEQ ID NO: 8 - protein, 120 aa, Artificial Sequence
  Synthetic Polypeptides
EVQLVQSGAEVKKPGASVKVSCKASGYTFTSHGISWVRQAPGQGLDWMGWISPYSGNTNYAQKLQGRVTMTTDTSTSTAYMELSSLRSEDTAVYYCARVGSGPYYYMDVWGQGTLVTVSS

SEQ ID NO: 9 - protein, 109 aa, Artificial Sequence
  Synthetic Polypeptides
QSALTQPRSVSGSPGQSVTISCTGTSSSVGDSIYVSWYQQHPGKAPKLMLYDVTKRPSGVPDRFSGSKSGNTASLTISGLQAEDEADYYCCSYAGTDTLFGGGTKVTVL

SEQ ID NO: 10 - protein, 109 aa, Artificial Sequence
  Synthetic Polypeptides
QSALTQPRSVSGSPGQSVTISCTGTSSSVGDSIYVSWYQQHPGKAPKLMLYDVTKRPSGVPDRFSGSKSGNTASLTISGLQAEDEADYYCYSYAGTDTLFGGGTKVTVL

SEQ ID NO: 11 - protein, 330 aa, Artificial Sequence
  Synthetic Polypeptides
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 330 aa, Artificial Sequence
  Synthetic Polypeptides
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 330 aa, Artificial Sequence
  Synthetic Polypeptides
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 14 - protein, 330 aa, Artificial Sequence
  Synthetic Polypeptides
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 15 - protein, 215 aa, Artificial Sequence
  Synthetic Polypeptides
QSALTQPRSVSGSPGQSVTISCTGTSSSVGDSIYVSWYQQHPGKAPKLMLYDVTKRPSGVPDRFSGSKSGNTASLTISGLQAEDEADYYCYSYAGTDTLFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 450 aa, Artificial Sequence
  Synthetic Polypeptides
EVQLVQSGAEVKKPGASVKVSCKASGYTFTSHGISWVRQAPGQGLDWMGWISPYSGNTNYAQKLQGRVTMTTDTSTSTAYMELSSLRSEDTAVYYCARVGSGPYYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 17 - protein, 450 aa, Homo sapiens
MQPPRERLVVTGRAGWMGMGRGAGRSALGFWPTLAFLLCSFPAATSPCKILKCNSEFWSATSGSHAPASDDTPEFCAALRSYALCTRRTARTCRGDLAYHSAVHGIEDLMSQHNCSKDGPTSQPRLRTLPPAGDSQERSDSPEICHYEKSFHKHSATPNYTHCGLFGDPHLRTFTDRFQTCKVQGAWPLIDNNYLNVQVTNTPVLPGSAATATSKLTIIFKNFQECVDQKVYQAEMDELPAAFVDGSKNGGDKHGANSLKITEKVSGQHVEIQAKYIGTTIVVRQVGRYLTFAVRMPEEVVNAVEDWDSQGLYLCLRGCPLNQQIDFQAFHTNAEGTGARRLAAASPAPTAPETFPYETAVAKCKEKLPVEDLYYQACVFDLLTTGDVNFTLAAYYALEDVKMLHSNKDKLHLYERTRDLPGRAAAGLPLAPRPLLGALVPLLALLPVFC

SEQ ID NO: 18 - protein, 122 aa, Homo sapiens
PCKILKCNSEFWSATSGSHAPASDDTPEFCAALRSYALCTRRTARTCRGDLAYHSAVHGIEDLMSQHNCSKDGPTSQPRLRTLPPAGDSQERSDSPEICHYEKSFHKHSATPNYTHCGLFGD

SEQ ID NO: 19 - protein, 23 aa, Homo sapiens
PCKILKCNSEFWSATSGSHAPAS

SEQ ID NO: 20 - protein, 6 aa, Artificial Sequence
  Synthetic Polypeptides
HHHHHH

SEQ ID NO: 21 - protein, 5 aa, Artificial Sequence
  Synthetic Polypeptides
DDDDK

SEQ ID NO: 22 - protein, 6 aa, Artificial Sequence
  Synthetic Polypeptides
ADDDDK

(19) United States
(12) Patent Application Publication
(10) Pub. No.: US 2022/0025031 A1
(43) Pub. Date: Jan. 27, 2022

(54) METHODS FOR TREATING SPINAL CORD INJURY AND PAIN

(71) Applicants: AbbVie Inc., North Chicago, IL (US); AbbVie Deutschland GmbH & Co. KG, Wiesbaden (DE)

(72) Inventors: Bernhard Klaus MUELLER, Ludwigshafen (DE); Peer B. JACOBSON, North Chicago, IL (US)

(21) Appl. No.: 17/196,425

(22) Filed: Mar. 9, 2021

Related U.S. Application Data
(63) Continuation of application No. 16/710,757, filed on Dec. 11, 2019, now abandoned.
Application No. 16/710,757 is a continuation of application No. 15/609,703, filed on May 31, 2017, now abandoned.
(60) Provisional application No. 62/344,233, filed on Jun. 1, 2016.

(51) Int. Cl. C07K 16/22; A61P 25/02; A61P 25/28

(52) U.S. Cl. CPC C07K 16/22; A61P 25/02; A61K 2039/505; C07K 2317/56; C07K 2317/565; A61P 25/28

(57) ABSTRACT

Disclosed herein are anti-RGMa antibodies and methods of using these antibodies to treat spinal cord injury, including promoting axonal regeneration, functional recovery, or both and to treat pain, including neuropathic pain arising from spinal cord injury.

### RELATED APPLICATIONS

This application is a continuation of U.S. patent application Ser. No. 16/710,757, filed Dec. 11, 2019, now abandoned, which is a continuation of U.S. patent application Ser. No. 15/609,703, filed May 31, 2017, now abandoned, which claims the benefit of U.S. Patent Application Ser. No. 62/344,233, filed Jun. 1, 2016, the contents of all of which are fully incorporated herein by reference.

### SEQUENCE LISTING

This application contains a Sequence Listing which has been submitted in ASCII format via EFS-Web and is hereby incorporated by reference in its entirety. Said ASCII copy, created on Mar. 8, 2021, is named 12303USC1_SequenceListing.txt and is 28,442 bytes in size.

### TECHNICAL FIELD

The present invention relates to anti-RGMa antibodies and methods of using these antibodies to treat spinal cord injury and/or pain, including neuropathic pain arising from spinal cord injury or other causes.

### BACKGROUND

Spinal cord injury (SCI) is a devastating condition with great personal and societal costs. Despite advances in clinical care, currently there is no effective treatment for major SCI. Following the initial trauma, there is a cascade of molecular and degenerative events including apoptosis, ischemia, excitotoxicity, and the upregulation of inhibitory molecules. Neuronal death and inhibition of axonal regeneration limit neurological recovery following injury. Injured CNS axons have a limited capacity to regenerate and often retract away from the injury site or undergo secondary axonal degeneration due to intrinsic mechanisms and the inhibitory environment of the injured spinal cord.

SCI represents a medical indication characterized by a high medical need with a worldwide annual incidence of 15-40 cases per million. The most common causes of SCI include motor vehicle accident, working accident, sporting/reaction accident, fall, and violence. In the United States, there are an estimated 12,000 new cases of SCI each year.

Most spinal cord injuries are contusion or compression injuries and the primary injury is usually followed by secondary injury mechanisms (e.g., inflammatory mediators such as cytokines and chemokines) that worsen the initial injury and result in significant enlargement of the lesion area, sometimes more than 10-fold.

Many SCIs are a result of the spinal cord being compressed, rather than cut. Insult to the spinal cord often results in vertebrae, nerve and blood vessel damage. Bleeding, fluid accumulation, and swelling can occur inside the spinal cord or outside the spinal cord but within the vertebral canal. The pressure from the surrounding bone and meninges structure can further damage the spinal cord. Moreover, edema of the cord itself can additionally accelerate secondary tissue loss. There is considerable evidence that the primary mechanical injury initiates a cascade of secondary injury mechanisms including excessive excitatory neurotransmitter accumulation; edema formation; electrolyte shifts, including increased intracellular calcium; free radical production, especially oxidant-free radicals; and eicosanoid production. Therefore, certain SCIs can be viewed as a two-step process. The primary injury is mechanical, resulting from impact, compression or some other insult to the spinal column. The secondary injury is cellular and biochemical, wherein cellular/molecular reactions cause tissue destruction.

The inflammatory response occurring after SCI is one of the main contributors to secondary damage. Glial cells (microglia and astrocytes) and macrophages play a key role during the course of the inflammatory response after SCI. Apart from secondary injury, reactive glia and macrophages contribute to the failure of axon regeneration in the CNS. Reactive astrocytes, for instance, synthesize proteoglycans which have potent effects in inhibiting axonal outgrowth in the CNS. Microglia and macrophages also contribute to inhibit axonal outgrowth.

SCI is among the diseases with the highest risk of developing neuropathic pain with a prevalence of up to 50%. Neuropathic pain is one of the most debilitating consequences of SCI. Inflammation not only contributes to functional loss after SCI by inducing secondary damage and axon repulsion, but also contributes to the development of neuropathic pain.

Certain animal models (e.g., spinal cord hemi-section) may not induce significant trauma typically associated with majority of clinical spinal cord injuries. Moreover, spinal edema is likely minimal in these models. As such, these models may not be representative of the majority of clinical spinal cord injuries.

### SUMMARY

In one aspect, the present disclosure provides a method of treating a spinal cord injury in a subject in need thereof. In certain embodiments, the spinal cord injury is a compression, contusion, or impact injury.

In another aspect, the present disclosure provides a method of promoting axonal regeneration, functional recovery, or both in a subject having a spinal cord injury. In certain embodiments, the functional recovery is assessed by a neurobehavioral test. In certain embodiments, the spinal cord injury is a compression, contusion, or impact injury.

In yet another aspect the present disclosure provides a method treating pain in a subject in need thereof. In certain embodiments, the pain is neuropathic pain, such as neuropathic pain arising from a spinal cord injury. In certain embodiments, the spinal cord injury is a compression, contusion, or impact injury.

The methods disclosed herein comprise administering a therapeutically effective amount of an antibody or antigen-binding fragment thereof that specifically binds Repulsive Guidance Molecule A (RGMa), wherein the antibody or antigen binding fragment comprises:

(a) a variable heavy chain comprising a complementarity determining region (VH CDR)-1 comprising an amino acid sequence of SEQ ID NO:1, a VH CDR-2 comprising an amino acid sequence of SEQ ID NO:2, and a VH CDR-3 comprising an amino acid sequence of SEQ ID NO:3; and

(b) a variable light chain comprising a complementarity determining region (VL CDR)-1 comprising an amino acid sequence of SEQ ID NO:4, a VL CDR-2 comprising an amino acid sequence of SEQ ID NO:5, and a VL CDR-3 comprising an amino acid sequence selected from the group consisting of SEQ ID NO:6 and SEQ ID NO:7. In certain embodiments, the VL CDR-3 comprises an amino acid sequence of SEQ ID NO:6. In certain other embodiments, the VL CDR-3 comprises an amino acid sequence of SEQ ID NO:7. In certain embodiments, the variable heavy chain comprises an amino acid sequence of SEQ ID NO: 8 and the variable light chain comprises an amino acid sequence of SEQ ID NO: 9. In certain other embodiments, the variable heavy chain comprises an amino acid sequence of SEQ ID NO: 8 and the variable light chain comprises an amino acid sequence of SEQ ID NO: 10. In certain embodiments, the antibody comprises a constant region comprising an amino acid sequence selected from the group consisting of SEQ ID NO: 11, SEQ ID NO: 12, SEQ ID NO: 13, and SEQ ID NO: 14. In certain embodiments, the antibody comprises a heavy chain sequence of SEQ ID NO: 16 and a light chain sequence of SEQ ID NO: 15.

In certain embodiments, the antibody is selected from the group consisting of a human antibody, an immunoglobulin molecule, a disulfide linked Fv, a monoclonal antibody, an affinity matured antibody, a scFv, a chimeric antibody, a CDR-grafted antibody, a diabody, a humanized antibody, a multispecific antibody, a Fab, a dual specific antibody, a DVD, a Fab′, a bispecific antibody, a F(ab′)2, and a Fv. In certain particular embodiments, the antibody is a human antibody.

In certain embodiments, the antibody is a monoclonal antibody.

In certain embodiments, the antibody or antigen-binding fragment thereof is administered systemically. In certain particular embodiments, the antibody or antigen-binding fragment thereof is administered intravenously.

In certain embodiments, the antibody is administered within 24 hours of the spinal cord injury.

The present disclosure demonstrates that RGMa is upregulated in multiple cell types after a clinically relevant impact-compression SCI in rats. Importantly, the present disclosure also demonstrates that RGMa is similarly upregulated in the human spinal cord after injury. To neutralize inhibitory RGMa, a human monoclonal anti-RGMa antibody was systemically administered weekly in a clinically relevant rat model of acute thoracic SCI, and was detected in serum, CSF, and in tissue around the lesion site. Rats treated with an anti-RGMa antibody showed improved neurobehavioural recovery in open field locomotion, fewer footfall errors on the ladderwalk, and improved gait parameters. RGMa neutralization promoted neuronal survival via attenuated apoptosis. Furthermore, this strategy enhanced the plasticity of descending corticospinal tract axonal regeneration as demonstrated with anterograde tracing. Interestingly, RGMa neutralization also attenuated neuropathic pain responses and was associated with fewer activated microglia and reduced calcitonin gene-related peptide (CGRP) expression in the dorsal horn caudal to the lesion.

The present disclosure demonstrates that systemic administration of an anti-RGMa antibody improved neuromotor function in a very severe, thoracic non-human primate (NHP) SCI hemicompression model. A significant improvement in overall neuromotor function was observed following systemic administration of an anti-RGMa antibody.

These findings show the therapeutic potential of neutralizing inhibitory RGMa after SCI and, in particular, contusion or compression injuries.

### BRIEF DESCRIPTION OF DRAWINGS

FIG. 1A-1C illustrates RGMa expression in rat spinal cord. FIG. 1A shows RGMa in neurons. FIG. 1B shows RGMa in oligodendrocytes. FIG. 1C shows RGMa in astrocytes and microglia. RGMa is upregulated in the spinal cord after injury. After injury, perilesional neurons express RGMa (FIG. 1A). In the normal and injured rat spinal cord, oligodendrocytes express RGMa (FIG. 1B). After SCI, RGMa is expressed by astrocytes, and within CSPG scar-rich regions within and surrounding the lesion site (FIG. 1C). Activated microglia and macrophages also express RGMa (FIG. 1D).

FIG. 2A-2F illustrates RGMa expression in adult human spinal cord. FIG. 2A shows RGMa in uninjured human spinal cord (low magnification). FIG. 2B shows higher magnification of the boxed region labeled “B” in FIG. 2A. FIG. 2C shows higher magnification of the boxed region labeled “C” in FIG. 2A. FIG. 2D shows RGMa in injured human spinal cord, 3 days post-injury (low magnification). FIG. 2E shows higher magnification of the boxed region labeled “E” in FIG. 2D. FIG. 2F shows higher magnification of the boxed region labeled “F” in FIG. 2D. In the uninjured human spinal cord, RGMa is expressed at low levels (FIG. 2A-2C). In the injured human spinal cord (3 day post-injury) RGMa expression is upregulated (FIG. 2D-2F).

FIG. 3A-3C illustrates RGMa expression in mouse cortical neurons. FIG. 3A depicts a Western blot showing RGMa in mouse cortical neuron lysates. FIG. 3B depicts immunostaining of RGMa in cultured mouse primary cortical neurons. FIG. 3C depicts mouse cortical neurons after incubation with RGMa and either hIgG, AE12-1, or AE12-1-Y.

FIG. 4A is a schematic showing the study design. FIG. 4B is a graph showing antibody concentration in CSF sampled at 6 weeks post-SCI. FIG. 4C is a graph showing antibody concentration in serum obtained at 9 weeks post-SCI. FIG. 4D depicts immunostaining of rat spinal cord with anti-human IgG. Human IgG (red) was detected around blood vessels (RECA-1, green) and within scar tissue (CSPG, green).

FIG. 5A-5D illustrates functional recovery after SCI in rats treated with AE12-1, AE12-1-Y, human IgG, or PBS. FIG. 5A is a line graph showing scores on the open field Basso, Beattie and Bresnahan (BBB) locomotor test. FIG. 5B is a line graph showing motor subscore. FIG. 5C is a line graph showing hindlimb footfall errors on the ladderwalk.

FIG. 5D is a bar graph showing percentage of successful hind limb steps. Rats treated with monoclonal antibody AE12-1 showed significant improvement on the BBB relative to hIgG and PBS controls (FIG. 5A). AE12-1 and AE12-1Y treated rats showed higher motor subscores relative to controls but this was not statistically significant (FIG. 5B). Rats treated with AE12-1 showed significantly fewer hind limb footfall errors on the ladderwalk compared to PBS controls at 3 weeks post-SCI and a trend towards reduced errors at 6 weeks (FIG. 5C). At 6 weeks post-SCI, AE12-1 treated rats showed a significantly higher percentage of successful hind limb steps compared to control (FIG. 5D).

FIG. 6A shows representative footprints obtained from the CatWalk from a rat pre-SCI and from each group at 6 weeks post-SCI. FIG. 6B is a series of bar graphs showing the regularity index, hindlimb stride length, hindlimb swing speed, and hindlimb intensity values in rats treated with AE12-1, AE12-1-Y, human IgG, or PBS following SCI. Rats treated with both monoclonal antibodies showed significant improvement in the regularity index relative to control groups (FIG. 6B). The monoclonal antibody treated rats showed a trend towards improved hind limb stride length and swing speed (FIG. 6B). Rats injected with AE12-1 showed significantly higher hindlimb intensity values relative to controls (FIG. 6B).

FIG. 7A-7D illustrates neuronal survival in rats treated with AE12-1, AE12-1-Y, human IgG, or PBS. FIG. 7A is a low magnification image of parasagittal sections of injured spinal cord 9 weeks post-SCI. FIG. 7B is a bar graph showing the number of spared perilesional neurons at 9 weeks post-SCI. FIG. 7C depicts immunostaining of neurons at 7 hours post-SCI. Double-labeling with NeuN (green) and TUNEL (red) identified apoptotic neurons (arrows). FIG. 7D is a bar graph showing the average number of NeuN+/TUNEL+ cells counted per section at 7 hours post-SCI. Rats administered monoclonal antibodies AE12-1 or AE12-1Y show significantly higher perilesional neuronal sparing as compared to rats that received hIgG and PBS (FIG. 7B). The average number of NeuN+/TUNEL+ cells counted per section was significantly less in AE12-1 treated rats than in rats administered PBS vehicle (FIG. 7D).

FIG. 8A-8E illustrates axonal regeneration in rats treated with AE12-1, AE12-1-Y, human IgG, or PBS following SCI. FIG. 8A depicts low magnification images of spinal cord following anterograde axonal tracing with BDA. FIG. 8B is a bar graph showing the average maximal length of BDA labeled CST fibers. FIG. 8C is a bar graph showing the average number of axons/section. FIG. 8D is a bar graph the average maximal length of BDA labeled CST fibers at 4 or 6 weeks post-SCI. FIG. 8E is a bar graph the average number of axons/section at 4 or 6 weeks post-SCI. The average maximal length of BDA labeled CST fibers increased after AE12-1 and AE12-1Y treatments (FIG. 8B). The average number of axons/section quantitated shows a greater number of axons in injured rats treated with the monoclonal antibodies (FIG. 8C). The average axonal length was significantly greater at 6 weeks compared to 4 weeks in injured rats treated with AE12-1Y (FIG. 8D & FIG. 8E).

FIG. 9A-9G illustrates neuropathic pain and inflammatory responses in rats treated with AE12-1, AE12-1-Y, human IgG, or PBS following SCI. FIG. 9A is a bar graph depicting the percentage of adverse responses in to 2 g von Frey monofilaments. FIG. 9B is a bar graph depicting the percentage of adverse responses in to 4 g von Frey monofilaments.

FIG. 9C is a bar graph depicting tail flick latency in response to noxious skin. FIG. 9D depicts Iba-1+ microglia caudal to the lesion at T10. FIG. 9E depicts Iba-1+ microglia at level T10.

FIG. 9F depicts Iba-1+ microglia rostral to the lesion at T10. FIG. 9G depicts CGRP+ cells at level T10. At 6 weeks post-SCI, AE12-1 treated rats showed significantly fewer adverse responses to the 4 g stimulus relative to controls (FIG. 9B). At 2 and 6 weeks post-SCI, monoclonal antibody treated rats showed reduced withdrawal of the tail in response to noxious skin heating relative to controls (FIG. 9C). At level T10, significantly more Iba-1+ cells were counted in the dorsal horn in controls compared to normal cord (FIGS. 9D & 9E). Percent CGRP+ area was significantly reduced in AE12-1 and AE12-1Y treated rats relative to controls (FIG. 9G).

FIG. 10A-10C illustrates RGMa expression in the adult rat spinal cord after injury. FIG. 10A depicts RGMa immunostaining in the ventral horn gray matter in normal intact cord and at 1 week post-SCI. FIG. 10B depicts RGMa expression in ED-1+ regions after SCI. FIG. 10C depicts high magnification images showing RGMa expression in oligodendrocytes (CC1) in the spinal cord white matter of normal intact cord. Quantification of % RGMa+ area shows significant upregulation of RGMa expression in the adult rat spinal cord after SCI (FIG. 10A). RGMa expression is apparent in ED-1+ regions after SCI (FIG. 10B).

FIG. 11A-11D illustrates neuronal expression of RGMa and Neogenin in the adult human spinal cord. FIG. 11A depicts RGMa expression in anterior horn neurons in normal adult human spinal cord. FIG. 11B depicts an adjacent section stained with RGMa antibody pre-absorbed with RGMa peptide showing specificity of staining. FIG. 11C depicts Neogenin expression in anterior horn neurons in normal adult human spinal cord. FIG. 11D depicts an adjacent negative control section.

FIG. 12A-12B illustrates expression of the RGMa receptor Neogenin. FIG. 12A depicts Western blot of adult rat brain lysates showing Neogenin expression. FIG. 12B depicts cultured mouse cortical neurons (3 div; F-actin, green) expressing Neogenin (red).

FIG. 13 depicts rat weights pre-SCI and at 4 and 6 weeks post-SCI. Rat weight did not vary significantly between groups. Treatment did not alter rat weight.

FIG. 14A-14B illustrates cavitation in rats treated with AE12-1, AE12-1-Y, human IgG, or PBS following SCI. Neutralization of RGMa with monoclonal antibodies results in no significant difference in cavitation.

FIG. 15A-15C illustrates the effect of an anti-RGMa antibody on astrogliosis and scarring. FIG. 15A depicts GFAP immunoreactivity adjacent to the lesion at 9 weeks post-SCI. FIG. 15B depicts quantification of % GFAP+ area rostral to the lesion. FIG. 15C depicts % CSPG+ area at the lesion site. Quantification of % GFAP+ area shows a significant reduction in astrogliosis rostral to the lesion in AE12-1Y treated rats at 9 weeks post-SCI (FIG. 15B). AE12-1 and AE12-1Y treated rats show a trend towards reduced % CSPG+ area at the lesion site (FIG. 15C).

FIG. 16A-16B illustrates BDA labeling of CST. FIG. 16A depicts BDA staining of dorsal CST at level C4, shown in transverse orientation. FIG. 16B depicts in parasagittal orientation 3 mm rostral to the lesion, BDA labeled CST axons are bundled in the dorsal CST fiber tract.

FIG. 17A-17B illustrates 5HT fibers. FIG. 17A depicts 5HT immunoreactive fibers (arrows) caudal to the lesion. FIG. 17B depicts quantification of the mean number of 5HT+ axons caudal to the lesion binned into progressive distances caudally. 5HT+ axons caudal to the lesion were binned into progressive distances caudally. A significantly higher number of 5HT+ fibers were apparent in AE12-1 treated rats and rats injected with AE12-1Y showed a trend towards higher number of 5HT labeled axons (FIG. 17B).

FIG. 18A-18B illustrates microglia and macrophages in rats treated with AE12-1, AE12-1-Y, human IgG, or PBS following SCI. FIG. 18A depicts Iba-1 immunoreactivity caudal to the lesion. FIG. 18B depicts % Iba-1+ area rostral or caudal to the lesion site. Adjacent to the lesion at T8, there was no significant difference between groups in the % Iba-1+ area rostral or caudal to the lesion site (FIG. 18B).

FIG. 19A is a graphical representation of neuromotor scores for individual control animals (and an estimated central value curve) following SCI. FIG. 19B is a graphical representation of neuromotor scores for individual animals that received IV AE-12-1-Y-QL treatment (and an estimated central value curve) following SCI.

FIG. 20A is a bar graph depicting tissue integrity in extra-lesional regions as assessed by fractional anisotropy (FA) in control and IV AE12-1-Y-QL treated groups following SCI. FIG. 20B is a bar graph depicting tissue integrity in extra-lesional regions as assessed by magnetization transfer ratio (MTR) in control and IV AE12-1-Y-QL treated groups following SCI. Intravenous AE12-1-Y-QL demonstrated a greater preservation of tissue integrity in the extra-injury regions as compared to an IgG control group.

FIG. 21A and FIG. 21B depict the correlation between individual neuromotor scores (NMS) and individual FA values or individual MTR values, respectively. The FA and MTR values generally increase with improved neuromotor function.

FIG. 22A-22F are bar graphs depicting histopathological analysis of spinal cord sections. FIGS. 22A and 22D depict RGMa expression at the rostral and caudal level, respectively. FIGS. 22B and 22E depict ionized calcium binding adaptor molecule 1 (IBA) expression at the rostral and caudal level, respectively. FIGS. 22C and 22F depict Weil staining of myelin at the rostral and caudal level, respectively.

FIG. 23A-23B illustrates functional recovery after SCI in rats treated with AE12-1-Y-QL or IgG. FIG. 23A is a line graph showing scores on the open field Basso, Beattie and Bresnahan (BBB) locomotor test. FIG. 23B is a line graph showing motor subscore.

FIG. 24A-24D is a series of bar graphs showing the regularity index (FIG. 24A), hindlimb stride length (FIG. 24B), hindlimb swing speed (FIG. 24C), and hindlimb intensity values (FIG. 24D) in rats treated with AE12-1-Y-QL or IgG following SCI.

FIG. 25A-25C illustrates neuropathic pain and inflammatory responses in rats treated with AE12-1-Y-QL or IgG following SCI. FIG. 25A is a bar graph depicting the percentage of adverse responses in to 2 g von Frey monofilaments. FIG. 25B is a bar graph depicting the percentage of adverse responses in to 4 g von Frey monofilaments. FIG. 25C is a bar graph depicting tail flick latency in response to noxious skin.

### DETAILED DESCRIPTION

Provided herein are methods of treating a spinal cord injury, promoting axonal regeneration following a spinal cord injury, promoting functional recovery following a spinal cord injury, and treating pain, including neuropathic pain arising from a spinal cord injury, by administering to a patient in need thereof a therapeutically effective amount of one or more anti-RGMa antibodies.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“About” as used herein may refer to approximately a +/−10% variation from the stated value. It is to be understood that such a variation is always included in any given value provided herein, whether or not specific reference is made to it.

“Affinity Matured Antibody” is used herein to refer to an antibody with one or more alterations in one or more CDRs, which result in an improvement in the affinity (i.e. K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J. Immunol., 154(7): 3310-3319 (1995); and Hawkins et al, J. Mol. Biol., 226: 889-896 (1992). Selective mutation at selective mutagenesis positions and at contact or hypermutation positions with an activity-enhancing amino acid residue is described in U.S. Pat. No. 6,914,128 B1.

“Antibody” and “antibodies” as used herein refers to monoclonal antibodies, multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)2 fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA and IgY), class (for example, IgG1, IgG2, IgG3, IgG4, IgA1 and IgA2) or subclass. For simplicity sake, an antibody against an analyte is frequently referred to herein as being either an “anti-analyte antibody,” or merely an “analyte antibody” (e.g., an anti-RGMa antibody or an RGMa antibody).

“Antibody fragment” as used herein refers to a portion of an intact antibody comprising the antigen-binding site or variable region. The portion does not include the constant heavy chain domains (i.e. CH2, CH3 or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Bispecific antibody” is used herein to refer to a full-length antibody that is generated by quadroma technology (see Milstein et al., Nature, 305(5934): 537-540 (1983)), by chemical conjugation of two different monoclonal antibodies (see, Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences), and is monovalent for each antigen to which it binds.

“CDR” is used herein to refer to the “complementarity determining region” within an antibody variable sequence. There are three CDRs in each of the variable regions of the heavy chain and the light chain, which are designated “CDR1”, “CDR2”, and “CDR3”, for each of the variable regions. The term “CDR set” as used herein refers to a group of three CDRs that occur in a single variable region that binds the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains or fragments of antibodies. The derivative may also comprise at least one further compound, e.g. a protein domain, said protein domain being linked by covalent or non-covalent bonds. The linkage can be based on genetic fusion according to the methods known in the art. The additional domain present in the fusion protein comprising the antibody employed in accordance with the invention may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g. a cytokine or growth hormone), a chemical agent, a peptide, a protein or a drug, for example.

“Dual-specific antibody” is used herein to refer to a full-length antibody that can bind two different antigens (or epitopes) in each of its two binding arms (a pair of HC/LC) (see PCT publication WO 02/02773). Accordingly a dual-specific binding protein has two identical antigen binding arms, with identical specificity and identical CDR sequences, and is bivalent for each antigen to which it binds.

“Dual variable domain” is used herein to refer to two or more antigen binding sites on a binding protein, which may be divalent (two antigen binding sites), tetravalent (four antigen binding sites), or multivalent binding proteins. DVDs may be monospecific, i.e., capable of binding one antigen (or one specific epitope), or multispecific, i.e., capable of binding two or more antigens (i.e., two or more epitopes of the same target antigen molecule or two or more epitopes of different target antigens). A preferred DVD binding protein comprises two heavy chain DVD polypeptides and two light chain DVD polypeptides and is referred to as a “DVD immunoglobulin” or “DVD-Ig”. Such a DVD-Ig binding protein is thus tetrameric and reminiscent of an IgG molecule, but provides more antigen binding sites than an IgG molecule. Thus, each half of a tetrameric DVD-Ig molecule is reminiscent of one half of an IgG molecule and comprises a heavy chain DVD polypeptide and a light chain DVD polypeptide, but unlike a pair of heavy and light chains of an IgG molecule that provides a single antigen binding domain, a pair of heavy and light chains of a DVD-Ig provide two or more antigen binding sites.

Each antigen binding site of a DVD-Ig binding protein may be derived from a donor (“parental”) monoclonal antibody and thus comprises a heavy chain variable domain (VH) and a light chain variable domain (VL) with a total of six CDRs involved in antigen binding per antigen binding site. Accordingly, a DVD-Ig binding protein that binds two different epitopes (i.e., two different epitopes of two different antigen molecules or two different epitopes of the same antigen molecule) comprises an antigen binding site derived from a first parental monoclonal antibody and an antigen binding site of a second parental monoclonal antibody.

A description of the design, expression, and characterization of DVD-Ig binding molecules is provided in PCT Publication No. WO 2007/024715, U.S. Pat. No. 7,612,181, and Wu et al., Nature Biotech., 25: 1290-1297 (2007). A preferred example of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, but preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, but preferably 1. Such a DVD-Ig may comprise two such heavy chains and two such light chains, wherein each chain comprises variable domains linked in tandem without an intervening constant region between variable regions, wherein a heavy chain and a light chain associate to form tandem functional antigen binding sites, and a pair of heavy and light chains may associate with another pair of heavy and light chains to form a tetrameric binding protein with four functional antigen binding sites. In another example, a DVD-Ig molecule may comprise heavy and light chains that each comprise three variable domains (VD1, VD2, VD3) linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

In an embodiment, a DVD-Ig binding protein according to the invention not only binds the same target molecules bound by its parental monoclonal antibodies, but also possesses one or more desirable properties of one or more of its parental monoclonal antibodies. For example, such an additional property is an antibody parameter of one or more of the parental monoclonal antibodies. Antibody parameters that may be contributed to a DVD-Ig binding protein from one or more of its parental monoclonal antibodies include, but are not limited to, antigen specificity, antigen affinity, potency, biological function, epitope recognition, protein stability, protein solubility, production efficiency, immunogenicity, pharmacokinetics, bioavailability, tissue cross reactivity, and orthologous antigen binding.

A DVD-Ig binding protein binds at least one epitope of RGMa. Non-limiting examples of a DVD-Ig binding protein include a DVD-Ig binding protein that binds one or more epitopes of RGMa, a DVD-Ig binding protein that binds an epitope of a human RGMa and an epitope of a RGMa of another species (for example, mouse), and a DVD-Ig binding protein that binds an epitope of a human RGMa and an epitope of another target molecule (for example, VEGFR2 or VEGFR1).

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

“Framework” (FR) or “Framework sequence” as used herein may mean the remaining sequences of a variable region minus the CDRs. Because the exact definition of a CDR sequence can be determined by different systems (for example, see above), the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3, and FR4) on each chain, in which CDR1 is positioned between FR1 and FR2, CDR2 between FR2 and FR3, and CDR3 between FR3 and FR4. Without specifying the particular sub-regions as FR1, FR2, FR3, or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region.

Human heavy chain and light chain FR sequences are known in the art that can be used as heavy chain and light chain “acceptor” framework sequences (or simply, “acceptor” sequences) to humanize a non-human antibody using techniques known in the art. In one embodiment, human heavy chain and light chain acceptor sequences are selected from the framework sequences listed in publicly available databases such as V-base or in the international ImMunoGeneTics® (IMGT®) information system.

“Functional antigen binding site” as used herein may mean a site on a binding protein (e.g. an antibody) that is capable of binding a target antigen. The antigen binding affinity of the antigen binding site may not be as strong as the parent binding protein, e.g., parent antibody, from which the antigen binding site is derived, but the ability to bind antigen must be measurable using any one of a variety of methods known for evaluating protein, e.g., antibody, binding to an antigen. Moreover, the antigen binding affinity of each of the antigen binding sites of a multivalent protein, e.g., multivalent antibody, herein need not be quantitatively the same.

“Human antibody” as used herein may include antibodies having variable and constant regions derived from human germline immunoglobulin sequences. The human antibodies described herein may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

“Humanized antibody” is used herein to describe an antibody that comprises heavy and light chain variable region sequences from a non-human species (e.g. a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody can be selected from any class of immunoglobulins, including IgM, IgG, IgD, IgA, and IgE, and any isotype, including without limitation IgG1, IgG2, IgG3, and IgG4. A humanized antibody may comprise sequences from more than one class or isotype, and particular constant domains may be selected to optimize desired effector functions using techniques well-known in the art.

The framework regions and CDRs of a humanized antibody need not correspond precisely to the parental sequences, e.g., the donor antibody CDR or the consensus framework may be mutagenized by substitution, insertion, and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, 1987)). A “consensus immunoglobulin sequence” may thus comprise a “consensus framework region(s)” and/or a “consensus CDR(s)”. In a family of immunoglobulins, each position in the consensus sequence is occupied by the amino acid occurring most frequently at that position in the family. If two amino acids occur equally frequently, either can be included in the consensus sequence.

“Linking sequence” or “linking peptide sequence” refers to a natural or artificial polypeptide sequence that is connected to one or more polypeptide sequences of interest (e.g., full-length, fragments, etc.). The term “connected” refers to the joining of the linking sequence to the polypeptide sequence of interest. Such polypeptide sequences are preferably joined by one or more peptide bonds. Linking sequences can have a length of from about 4 to about 50 amino acids. Preferably, the length of the linking sequence is from about 6 to about 30 amino acids. Natural linking sequences can be modified by amino acid substitutions, additions, or deletions to create artificial linking sequences. Exemplary linking sequences include, but are not limited to: (i) Histidine (His) tags, such as a 6× His tag (SEQ ID NO: 20), which has an amino acid sequence of HHHHHH (SEQ ID NO: 20), are useful as linking sequences to facilitate the isolation and purification of polypeptides and antibodies of interest; (ii) Enterokinase cleavage sites, like His tags, are used in the isolation and purification of proteins and antibodies of interest. Often, enterokinase cleavage sites are used together with His tags in the isolation and purification of proteins and antibodies of interest. Various enterokinase cleavage sites are known in the art. Examples of enterokinase cleavage sites include, but are not limited to, the amino acid sequence of DDDDK (SEQ ID NO: 21) and derivatives thereof (e.g., ADDDDK (SEQ ID NO: 22), etc.); (iii) Miscellaneous sequences can be used to link or connect the light and/or heavy chain variable regions of single chain variable region fragments. Examples of other linking sequences can be found in Bird et al., Science 242: 423-426 (1988); Huston et al., PNAS USA 85: 5879-5883 (1988); and McCafferty et al., Nature 348: 552-554 (1990). Linking sequences also can be modified for additional functions, such as attachment of drugs or attachment to solid supports. In the context of the present disclosure, the monoclonal antibody, for example, can contain a linking sequence, such as a His tag, an enterokinase cleavage site, or both.

“Multivalent binding protein” is used herein to refer to a binding protein comprising two or more antigen binding sites (also referred to herein as “antigen binding domains”). A multivalent binding protein is preferably engineered to have three or more antigen binding sites, and is generally not a naturally occurring antibody. The term “multispecific binding protein” refers to a binding protein that can bind two or more related or unrelated targets, including a binding protein capable of binding two or more different epitopes of the same target molecule.

“Recombinant antibody” and “recombinant antibodies” refer to antibodies prepared by one or more steps, including cloning nucleic acid sequences encoding all or a part of one or more monoclonal antibodies into an appropriate expression vector by recombinant techniques and subsequently expressing the antibody in an appropriate host cell. The terms include, but are not limited to, recombinantly produced monoclonal antibodies, chimeric antibodies, humanized antibodies (fully or partially humanized), multi-specific or multi-valent structures formed from antibody fragments, bifunctional antibodies, heteroconjugate Abs, DVD-Ig's, and other antibodies as described in (i) herein. (Dual-variable domain immunoglobulins and methods for making them are described in Wu, C., et al., Nature Biotechnology, 25:1290-1297 (2007)). The term “bifunctional antibody,” as used herein, refers to an antibody that comprises a first arm having a specificity for one antigenic site and a second arm having a specificity for a different antigenic site, i.e., the bifunctional antibodies have a dual specificity.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Treat”, “treating” or “treatment” are each used interchangeably herein to describe reversing, alleviating, or inhibiting the progress of a disease, or one or more symptoms of such disease, to which such term applies. A treatment may be either performed in an acute or chronic way. The term also refers to reducing the severity of a disease or symptoms associated with such disease prior to affliction with the disease. Such reduction of the severity of a disease prior to affliction refers to administration of an antibody or pharmaceutical composition described herein to a subject that is not at the time of administration afflicted with the disease. “Treatment” and “therapeutically,” refer to the act of treating, as “treating” is defined above.

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically reactive fragment of an anti-RGMa antibody that differs from the corresponding fragment of anti-RGMa antibody in amino acid sequence but is still antigenically reactive and can compete with the corresponding fragment of anti-RGMa antibody for binding with RGMa. “Variant” also can be used to describe a polypeptide or a fragment thereof that has been differentially processed, such as by proteolysis, phosphorylation, or other post-translational modification, yet retains its antigen reactivity.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. ANTI-RGMA ANTIBODIES

Provided herein are methods of methods of treating a spinal cord injury, promoting axonal regeneration following a spinal cord injury, promoting functional recovery following a spinal cord injury, and treating pain, including neuropathic pain arising from a spinal cord injury, by administering to a patient in need thereof one or more anti-RGMa antibodies. The anti-RGMa antibodies for use in the methods described herein bind to RGMa, while minimizing or eliminating reactivity with Repulsive Guidance Molecule c (“RGMc”). Because antibodies raised against RGMa can often cross-react with RGMc and, at high intravenous doses may result in iron accumulation in hepatocytes, the specific binding of the herein described antibodies for RGMa is of therapeutic benefit. Further, the high selectivity of these antibodies offers large therapeutic dose windows or ranges for treatment.

a. RGMa-Recognizing Antibody

An antibody that can be used in the methods described herein, is an antibody that binds to RGMa, a fragment or variant thereof. Such antibodies are described, for example, in WO 2013112922, the entire contents of which are herein incorporated by reference. The antibody may be a fragment of the anti-RGMa antibody or a variant or a derivative thereof. The antibody may be a polyclonal or monoclonal antibody. The antibody may be a chimeric antibody, a single chain antibody, an affinity matured antibody, a human antibody, a humanized antibody, a fully human antibody or an antibody fragment, such as a Fab fragment, or a mixture thereof. Antibody fragments or derivatives may comprise F(ab′)₂, Fv or scFv fragments. The antibody derivatives can be produced by peptidomimetics. Further, techniques described for the production of single chain antibodies can be adapted to produce single chain antibodies.

Human antibodies may be derived from phage-display technology or from transgenic mice that express human immmunoglobulin genes. The human antibody may be generated as a result of a human in vivo immune response and isolated. See, for example, Funaro et al., BMC Biotechnology, 2008(8):85. Therefore, the antibody may be a product of the human and not animal repertoire. Because it is of human origin, the risks of reactivity against self-antigens may be minimized. Alternatively, standard yeast display libraries and display technologies may be used to select and isolate human anti-RGMa antibodies. For example, libraries of naïve human single chain variable fragments (scFv) may be used to select human anti-RGMa antibodies. Transgenic animals may be used to express human antibodies.

Humanized antibodies may be antibody molecules from non-human species antibody that binds the desired antigen having one or more complementarily determining regions (CDRs) from the non-human species and framework regions from a human immunoglobulin molecule.

The antibody may specifically bind to RGMa. In certain embodiments, the anti-RGMa antibody binds to an epitope located in the N-terminal region of RGMa.

The antibody may bind to SEQ ID NO: 17, SEQ ID NO: 18, SEQ ID NO: 19, or a fragment or variant thereof. The antibody may recognize and specifically bind an epitope present on a RGMa polypeptide or a variant as described above. The epitope may be SEQ ID NO:17 (full-length human RGMa), SEQ ID NO:18 (a human RGMa fragment which corresponds to amino acids 47-168 of SEQ ID NO:17), SEQ ID NO:19 (a human RGMa fragment), or a variant thereof, the sequences of which are provided below:

(SEQ ID NO: 17)
MQPPRERLVVTGRAGWMGMGRGAGRSALGFWPTLAFLLCSFPAATSPCKI
LKCNSEFWSATSGSHAPASDDTPEFCAALRSYALCTRRTARTCRGDLAYH
SAVHGIEDLMSQHNCSKDGPTSQPRLRTLPPAGDSQERSDSPEICHYEKS
FHKHSATPNYTHCGLFGDPHLRTFTDRFQTCKVQGAWPLIDNNYLNVQVT
NTPVLPGSAATATSKLTIIFKNFQECVDQKVYQAEMDELPAAFVDGSKNG
GDKHGANSLKITEKVSGQHVEIQAKYIGTTIVVRQVGRYLTFAVRMPEEV
VNAVEDWDSQGLYLCLRGCPLNQQIDFQAFHTNAEGTGARRLAAASPAPT
APETFPYETAVAKCKEKLPVEDLYYQACVFDLLTTGDVNFTLAAYYALED
VKMLHSNKDKLHLYERTRDLPGRAAAGLPLAPRPLLGALVPLLALLPVFC
(SEQ ID NO: 18)
PCKILKCNSEFWSATSGSHAPASDDTPEFCAALRSYALCTRRTARTCRGD
LAYHSAVHGIEDLMSQHNCSKDGPTSQPRLRTLPPAGDSQERSDSPEICH
YEKSFHKHSATPNYTHCGLFGD
(SEQ ID NO: 19)
PCKILKCNSEFWSATSGSHAPAS.

In certain embodiments, the RGMa-specific RGMa antibody may comprise SEQ ID NOs: 1, 2, 3, 4, 5, and 6; SEQ ID NOs: 1, 2, 3, 4, 5, and 7; SEQ ID NOs: 1, 2, 3, and 9; SEQ ID NOs: 1, 2, 3, and 10; SEQ ID NOs: 4, 5, 6, and 8; SEQ ID NOs: 4, 5, 7, and 8; SEQ ID NOs: 8 and 9; SEQ ID NOs: 8 and 10; SEQ ID NOs: 1, 2, 3, and 15; SEQ ID NOs: 4, 5, 6, and 16; SEQ ID NOs: 4, 5, 7, and 16; or SEQ ID NOs: 15 and 16.

Previous data suggested that the epitope for AE12-1 is located in the N-terminal region of RGMa. In certain embodiments, the antibody binds to an RGMa epitope within amino acids 47-168 of human RGMa. In certain embodiments, the antibody binds to an RGMa epitope within the amino acids set forth in SEQ ID NO: 18. In certain embodiments, the antibody binds to an RGMa epitope within amino acids 47-69 of human RGMa. In certain embodiments, the antibody binds to an RGMa epitope within the amino acids set forth in SEQ ID NO: 19.

(1) Antibody Structure

(a) Heavy Chain and Light Chain CDRs

The antibody may immunospecifically bind to RGMa (SEQ ID NO: 17), SEQ ID NO: 18, SEQ ID NO: 19, a fragment thereof, or a variant thereof and comprise a variable heavy chain and/or variable light chain shown in Table 1. The antibody may immunospecifically bind to RGMa, a fragment, derivative, or a variant thereof and comprise one or more of the heavy chain or light chain CDR sequences also shown in Table 1. The light chain of the antibody may be a kappa chain or a lambda chain. For example, see Table 1. Methods for making the antibodies shown in Table 1 are described in WO 2013/112922, the contents of which are herein incorporated by reference.

TABLE 1
List of Amino Acid Sequences of VH and VL Regions
of Anti-RGMa Monoclonal Antibodies AE12-1 and
AE12-1-Y.

| | SEQ ID | |
|---|---|---|
| PROTEIN REGION | NO. | SEQUENCE |
| AE12-1 (VH) CDR-H1; | 1 | SHGIS |
| AE12-1-Y (VH) CDR-H1 | | |
| AE12-1 (VH) CDR-H2; | 2 | WISPYSGNTNYAQKLQG |
| AE12-1-Y (VH) CDR-H2 | | |
| AE12-1 (VH) CDR-H3; | 3 | VGSGPYYYMDV |
| AE12-1-Y (VH) CDR-H3 | | |
| AE12-1 (VL) CDR-L1; | 4 | TGTSSSVGDSIYVS |
| AE12-1-Y (VL) CDR-L1 | | |
| AE12-1 (VL) CDR-L2; | 5 | DVTKRPS |
| AE12-1-Y (VL) CDR-L2 | | |
| AE12-1 (VL) CDR-L3; | 6 | CSYAGTDTL |
| AE12-1-Y (VL) CDR-L3 | 7 | YSYAGTDTL |
| AE12-1 (VH) | 8 | EVQLVQSGAEVKKPGASVKVS |
| AE12-1-Y (VH) | | CKASGYTFTSHGISWVRQAPG |
| | | QGLDWMGWISPYSGNTNYAQK |
| | | LQGRVTMTTDTSTSTAYMELS |
| | | SLRSEDTAVYYCARVGSGPYY |
| | | YMDVWGQGTLVTVSS |
| AE12-1 (VL) | 9 | QSALTQPRSVSGSPGQSVTIS |
| | | CTGTSSSVGDSIYVSWYQQHP |
| | | GKAPKLMLYDVTKRPSGVPDR |
| | | FSGSKSGNTASLTISGLQAED |
| | | EADYYCCSYAGTDTLFGGGTK |
| | | VTVL |
| AE12-1-Y (VL) | 10 | QSALTQPRSVSGSPGQSVTIS |
| | | CTGTSSSVGDSIYVSWYQQHP |
| | | GKAPKLMLYDVTKRPSGVPDR |
| | | FSGSKSGNTASLTISGLQAED |
| | | EADYYCYSYAGTDTLFGGGTK |
| | | VTVL |

The antibody or variant or derivative thereof may contain one or more amino acid sequences that are greater than 95%, 90%, 85%, 80%, 75%, 70%, 65%, 60%, 55%, or 50% identical to one or more of SEQ ID NOs:1-10 or 15-16. The antibody or variant or derivative thereof may be encoded by one or more nucleic acid sequences that are greater than 95%, 90%, 85%, 80%, 75%, 70%, 65%, 60%, 55%, or 50% identical to one or more of SEQ ID NOs:1-10 or 15-16. Polypeptide identity and homology can be determined, for example, by the algorithm described in the report: Wilbur, W. J. and Lipman, D. J. Proc. Natl. Acad. Sci. USA 80, 726-730 (1983).

The antibody may be an IgG, IgE, IgM, IgD, IgA and IgY molecule class (for example, IgG1, IgG2, IgG3, IgG4, IgA1 and IgA2) or subclass. For example, the antibody may be an IgG1 molecule having the following constant region sequence:

(SEQ ID NO: 11)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

The above constant region in SEQ ID NO: 11 contains two (2) mutations of the wildtype constant region sequence at positions 234 and 235. Specifically, these mutations are leucine to alanine changes at each of positions 234 and 235 (which are referred to as the “LLAA” mutations). These mutations are shown above in bold and underlining. The purpose of these mutations is to eliminate the effector function.

Alternatively, an IgG1 molecule can have the above constant region sequence (SEQ ID NO: 11) containing one or more mutations. For example, the constant region sequence of SEQ ID NO: 11 may containing a mutation at amino acid 250 where threonine is replaced with glutamine (SEQ ID NO: 12), a mutation at amino acid 428 where methionine is replaced with leucine (SEQ ID NO: 13) or mutations at amino acid 250 where threonine is replaced with glutamine and a mutation at amino acid 428 where methionine is replaced with leucine (SEQ ID NO: 14) as shown below in Table 2.

TABLE 2

| Amino | SEQ | |
|---|---|---|
| acid | ID | |
| Mutation | NO: | SEQUENCE |
| None | 11 | ASTKGPSVFPLAPSSKSTSGGTAALGCLVKDYFPEPV |
| | | TVSWNSGALTSGVHTFPAVLQSSGLYSLSSVVTVPSS |
| | | SLGTQTYICNVNHKPSNTKVDKKVEPKSCDKTHTCPP |
| | | CPAPEAAGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKA |
| | | KGQPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPGK |
| T250Q | 12 | ASTKGPSVFPLAPSSKSTSGGTAALGCLVKDYFPEPV |
| | | TVSWNSGALTSGVHTFPAVLQSSGLYSLSSVVTVPSS |
| | | SLGTQTYICNVNHKPSNTKVDKKVEPKSCDKTHTCPP |
| | | CPAPEAAGGPSVFLFPPKPKDQLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKA |
| | | KGQPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVMHEALHNHYTQKSLSLSPGK |
| M428L | 13 | ASTKGPSVFPLAPSSKSTSGGTAALGCLVKDYFPEPV |
| | | TVSWNSGALTSGVHTFPAVLQSSGLYSLSSVVTVPSS |
| | | SLGTQTYICNVNHKPSNTKVDKKVEPKSCDKTHTCPP |
| | | CPAPEAAGGPSVFLFPPKPKDTLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKA |
| | | KGQPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVLHEALHNHYTQKSLSLSPGK |
| T250Q | 14 | ASTKGPSVFPLAPSSKSTSGGTAALGCLVKDYFPEPV |
| and | | TVSWNSGALTSGVHTFPAVLQSSGLYSLSSVVTVPSS |
| M428L | | SLGTQTYICNVNHKPSNTKVDKKVEPKSCDKTHTCPP |
| | | CPAPEAAGGPSVFLFPPKPKDQLMISRTPEVTCVVVD |
| | | VSHEDPEVKFNWYVDGVEVHNAKTKPREEQYNSTYRV |
| | | VSVLTVLHQDWLNGKEYKCKVSNKALPAPIEKTISKA |
| | | KGQPREPQVYTLPPSREEMTKNQVSLTCLVKGFYPSD |
| | | IAVEWESNGQPENNYKTTPPVLDSDGSFFLYSKLTVD |
| | | KSRWQQGNVFSCSVLHEALHNHYTQKSLSLSPGK |

Alternatively, an IgG1 molecule can contain a heavy chain comprising: AE12-1-Y (VH) CDR-H1 (SEQ ID NO: 1), AE12-1-Y (VH) CDR-H2 (SEQ ID NO: 2), AE12-1-Y (VH) CDR-H3 (SEQ ID NO: 3) and a light chain comprising: AE12-1-Y (VL) CDR-L1 (SEQ ID NO: 4), AE12-1-Y (VL) CDR-L2 (SEQ ID NO: 5) and AE12-1-Y (VL) CDR-L3 (SEQ ID NO: 7) and a constant sequence of SEQ ID NO: 14 as shown below in Table 3 (this antibody is referred to as AE12-1-Y-QL and has a light chain sequence of SEQ ID NO: 15 and a heavy chain sequence of SEQ ID NO: 16).

TABLE 3

| PROTEIN | SEQ ID | |
|---|---|---|
| REGION | NO: | SEQUENCE |
| AE12-1-Y- | 15 | QSALTQPRSVSGSPGQSVTISCTGTSSSVG |
| QL Light | | DSIYVSWYQQHPGKAPKLMLYDVTKRPSGV |
| chain | | PDRFSGSKSGNTASLTISGLQAEDEADYYC |
| (CDR's | | YSYAGTDTLFGGGTKVTVLGQPKAAPSVTL |
| underlined | | FPPSSEELQANKATLVCLISDFYPGAVTVA |
| and | | WKADSSPVKAGVETTTPSKQSNNKYAASSY |
| mutations | | LSLTPEQWKSHRSYSCQVTHEGSTVEKTVA |
| bolded) | | PTECS* |
| AE12-1-Y- | 16 | EVQLVQSGAEVKKPGASVKVSCKASGYTFT |
| QL Heavy | | SHGISWVRQAPGQGLDWMGWISPYSGNTNY |
| chain | | AQKLQGRVTMTTDTSTSTAYMELSSLRSED |
| (CDR's | | TAVYYCARVGSGPYYYMDVWGQGTLVTVSS |
| underlined | | ASTKGPSVFPLAPSSKSTSGGTAALGCLVK |
| and | | DYFPEPVTVSWNSGALTSGVHTFPAVLQSS |
| mutations | | GLYSLSSVVTVPSSSLGTQTYICNVNHKPS |
| bolded) | | NTKVDKKVEPKSCDKTHTCPPCPAPEAAGG |
| | | PSVFLFPPKPKDQLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSREE |
| | | MTKNQVSLTCLVKGFYPSDIAVEWESNGQP |
| | | ENNYKTTPPVLDSDGSFFLYSKLTVDKSRW |
| | | QQGNVFSCSVLHEALHNHYTQKSLSLSPGK* |

### 3. PHARMACEUTICAL COMPOSITIONS

The antibody may be a component in a pharmaceutical composition. The pharmaceutical composition may also contain a pharmaceutically acceptable carrier. The pharmaceutical compositions comprising antibodies described herein are for use in treating spinal cord injury, particularly promoting axonal regeneration, functional recovery, or both. The pharmaceutical compositions comprising antibodies described herein are also for use in treating pain, including, but not limited to, neuropathic pain arising from spinal cord injury. In a specific embodiment, a composition comprises one or more antibodies described herein. In accordance with these embodiments, the composition may further comprise of a carrier, diluent or excipient.

The antibodies described herein can be incorporated into pharmaceutical compositions suitable for administration to a subject. Typically, the pharmaceutical composition comprises an antibody described herein (such as, for example, AE-12-1, AE-12-1-Y, or AE-12-1-Y-QL) and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody.

In a further embodiment, the pharmaceutical composition comprises at least one additional therapeutic agent for treating a spinal cord injury or treating pain, including, but not limited to, neuropathic pain arising from spinal cord injury.

Various delivery systems are known and can be used to administer one or more antibodies described herein or the combination of one or more antibodies described herein e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent include, but are not limited to, parenteral administration (e.g., intradermal, intramuscular, intraperitoneal, intravenous, intrathecal and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO97/32572; WO97/44013; WO98/31346; and WO99/66903, each of which is incorporated herein by reference in their entireties. In one embodiment, an antibody described herein, combination therapy, or a composition described herein is administered using Alkermes AIR® pulmonary drug delivery technology (Alkermes, Inc., Cambridge, Mass.). In a specific embodiment, prophylactic or therapeutic agents of the antibodies described herein are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the antibodies described herein locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies described herein is administered locally to the affected area to a subject to prevent, treat, manage, and/or ameliorate a disorder or a symptom thereof. In another embodiment, an effective amount of one or more antibodies described herein is administered locally to the affected area in combination with an effective amount of one or more therapies (e.g., one or more prophylactic or therapeutic agents) other than an antibody described herein to a subject to prevent, treat, manage, and/or ameliorate a disorder or one or more symptoms thereof.

In certain embodiments, intrathecal administration may be ruled out as a treatment option (e.g., during early stages of injury if edema impedes CSF flow).

A pharmaceutical composition is formulated to be compatible with its intended route of administration. Examples of routes of administration include, but are not limited to, parenteral, e.g., intravenous, intrathecal, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

The method described herein may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The methods described herein may additionally comprise of administration of compositions formulated as depot preparations. Such long acting formulations may be administered by implantation (e.g., subcutaneously, intrathecally or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods described herein encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the methods described herein also contemplate that one or more of the antibodies or pharmaceutical compositions described herein are packaged in a hermetically sealed container such as an ampoule or sachette indicating the quantity of the antibody. In one embodiment, one or more of the antibodies, or pharmaceutical compositions described herein are supplied as a dry sterilized lyophilized powder or water free concentrate in a hermetically sealed container and can be reconstituted (e.g., with water or saline) to the appropriate concentration for administration to a subject. In one embodiment, one or more of the antibodies or pharmaceutical compositions described herein are supplied as a dry sterile lyophilized powder in a hermetically sealed container at a unit dosage of at least 5 mg, for example at least 10 mg, at least 15 mg, at least 25 mg, at least 35 mg, at least 45 mg, at least 50 mg, at least 75 mg, or at least 100 mg. The lyophilized antibodies or pharmaceutical compositions described herein should be stored at between 2° C. and 8° C. in its original container and the antibodies, or pharmaceutical compositions described herein should be administered within 1 week, for example within 5 days, within 72 hours, within 48 hours, within 24 hours, within 12 hours, within 6 hours, within 5 hours, within 3 hours, or within 1 hour after being reconstituted. In an alternative embodiment, one or more of the antibodies or pharmaceutical compositions described herein is supplied in liquid form in a hermetically sealed container indicating the quantity and concentration of the antibody. In a further embodiment, the liquid form of the administered composition is supplied in a hermetically sealed container at least 0.25 mg/ml, for example at least 0.5 mg/ml, at least 1 mg/ml, at least 2.5 mg/ml, at least 5 mg/ml, at least 8 mg/ml, at least 10 mg/ml, at least 15 mg/ml, at least 25 mg/ml, at least 50 mg/ml, at least 75 mg/ml or at least 100 mg/ml. The liquid form should be stored at between 2° C. and 8° C. in its original container.

The antibodies described herein can be incorporated into a pharmaceutical composition suitable for parenteral administration. In one aspect, antibodies will be prepared as an injectable solution containing 0.1-500 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the tonicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising the antibodies described herein prepared as an injectable solution for parenteral administration, can further comprise an agent useful as an adjuvant, such as those used to increase the absorption, or dispersion of the antibody. A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e. greater than 1 ml) with less pain and discomfort, and minimum incidence of injection site reactions. (See International Appln. Publication No. WO 04/078140 and U.S. Patent Appln. Publication No. US2006104968, incorporated herein by reference.)

The compositions described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Compositions can be in the form of injectable or infusible solutions, such as compositions similar to those used for passive immunization of humans with other antibodies. In one embodiment, the antibody is administered by intravenous infusion or injection. In another embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., a binding protein, e.g. an antibody described herein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, methods of preparation comprise vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The antibodies described herein can be administered by a variety of methods known in the art. For example, the route/mode of administration may be subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody described herein may be orally administered, for example, with an inert diluent or an assimilable edible carrier. The antibody (and other ingredients, if desired) may also be enclosed in a hard or soft shell gelatin capsule, compressed into tablets, or incorporated directly into the subject's diet. For oral therapeutic administration, the antibody may be incorporated with excipients and used in the form of ingestible tablets, buccal tablets, troches, capsules, elixirs, suspensions, syrups, wafers, and the like. To administer an antibody described herein by other than parenteral administration, it may be necessary to coat the antibody with, or co-administer the antibody with, a material to prevent its inactivation.

Supplementary active compounds can also be incorporated into the compositions. In certain embodiments, an antibody described herein is co-formulated with and/or co-administered with one or more additional therapeutic agents that are useful for treating disorders or diseases described herein. For example, an anti-RGMa antibody described herein may be co-formulated and/or co-administered with one or more additional antibodies that bind other targets (e.g., antibodies that bind other soluble antigens or that bind cell surface molecules). Furthermore, one or more antibodies described herein may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, an antibody described herein is linked to a half-life extending vehicle known in the art. Such vehicles include, but are not limited to, the Fc domain, polyethylene glycol, and dextran. Such vehicles are described, e.g., in U.S. application Ser. No. 09/428,082 and published PCT Application No. WO 99/25044, which are hereby incorporated by reference for any purpose.

It should be understood that the antibodies described herein can be used alone or in combination with one or more additional agents, e.g., a therapeutic agent (for example, a small molecule or biologic), said additional agent being selected by the skilled artisan for its intended purpose.

It should further be understood that the combinations are those combinations useful for their intended purpose. The agents set forth above are for illustrative purposes and not intended to be limiting. The combinations can comprise an antibody and at least one additional agent selected from the lists below. The combination can also include more than one additional agent, e.g., two or three additional agents if the combination is such that the formed composition can perform its intended function.

The pharmaceutical compositions may include a “therapeutically effective amount” or a “prophylactically effective amount” of an antibody. A “therapeutically effective amount” refers to an amount effective, at dosages and for periods of time necessary, to achieve the desired therapeutic result. A therapeutically effective amount of the antibody may be determined by a person skilled in the art and may vary according to factors such as the disease state, age, sex, and weight of the individual, and the ability of the antibody to elicit a desired response in the individual. A therapeutically effective amount is also one in which toxic or detrimental effects, if any, of the antibody are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of the antibody is a dose of between 0.1 and 200 mg/kg, for example between 0.1 and 100 mg/kg, between 5 and 50 mg/kg, or between 10 and 25 mg/kg. The therapeutically or prophylactically effective amount of the antibody may be between 1 and 200 mg/kg, 10 and 200 mg/kg, 20 and 200 mg/kg, 50 and 200 mg/kg, 75 and 200 mg/kg, 100 and 200 mg/kg, 150 and 200 mg/kg, 50 and 100 mg/kg, 5 and 10 mg/kg, or 1 and 10 mg/kg. It is to be noted that dosage values may vary with the type and severity of the condition to be alleviated. Further, the antibody dose may be determined by a person skilled in the art and may vary according to factors such as the disease state, age, sex, and weight of the individual, and the ability of the antibody to elicit a desired response in the individual. The dose is also one in which toxic or detrimental effects, if any, of the antibody are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### 4. METHODS OF TREATMENT

a. Spinal Cord Injury (SCI)

In any subject, an assessment may be made as to whether the subject has, or is at risk of having, a spinal cord injury. The assessment may indicate an appropriate course of therapy, such as preventative therapy, maintenance therapy, or modulative therapy. Accordingly, provided herein is a method of treating, preventing, modulating, or attenuating a spinal cord injury by administering a therapeutically effective amount of one or more of the antibodies described herein (such as, for example, antibody AE12-1, AE12-1-Y, or AE12-1-Y-QL). The antibody may be administered to a subject in need thereof. The antibody may be administered in a therapeutically effective amount.

In one embodiment, a cause of the spinal cord injury is a motor vehicle accident, fall, violence, sports injury, vascular disorder, tumor, infectious disease, spondylosis, latrogenic injury (especially after spinal injections and epidural catheter placement), vertebral fracture secondary to osteoporosis, or developmental disorder.

In certain embodiments, the spinal cord injury can result from, e.g., blunt force trauma, compression, displacement, or the like. In certain embodiments, the spinal cord is completely severed. In certain other embodiments, the spinal cord is damaged, e.g., partially severed, but not completely severed. In other embodiments, the spinal cord is compressed, e.g., through damage to the bony structure of the spinal column, displacement of one or more vertebrae relative to other vertebrae, inflammation or swelling of adjacent tissues, or the like.

Spinal cord injury includes conditions known as tetraplegia (formerly known as quadriplegia) and paraplegia. Thus, some embodiments of the method of treatment of spinal cord injury provided herein include treating a tetraplegic or paraplegic patient.

Tetraplegia refers to injury to the spinal cord in the cervical region, characterized by impairment or loss of motor and/or sensory function in the cervical segments of the spinal cord due to damage of neural elements within the spinal canal. Tetraplegia results in impairment of function in the arms as well as in the trunk, legs and pelvic organs. It does not include brachial plexus lesions or injury to peripheral nerves outside the neural canal.

Paraplegia refers to impairment or loss of motor and/or sensory function in the thoracic, lumbar or sacral (but not cervical) segments of the spinal cord, secondary to damage of neural elements within the spinal canal. With paraplegia, arm functioning is spared, but, depending on the level of injury, the trunk, legs and pelvic organs may be involved. The term is used in referring to cauda equina and conus medullaris injuries, but not to lumbosacral plexus lesions or injury to peripheral nerves outside the neural canal.

In one embodiment, the spinal cord injury is at one or more of the cervical vertebrae. In another embodiment, the spinal cord injury is at one or more of the thoracic vertebrae. In another embodiment, the spinal cord injury is at one or more of the lumbar vertebrae. In another embodiment, the spinal cord injury is at one or more of the sacral vertebrae. In certain embodiments, the spinal cord injury is at vertebra C1, C2, C3, C4, C5, C6 or C7; or at vertebra T1, T2, T3, T4, T5, T6, T7, T8, T9, T10, T11 or T12; or at vertebra L1, L2, L3, L4 or L5. In certain other embodiments, the spinal cord injury is to a spinal root exiting the spinal column between C1 and C2; between C2 and C3; Between C3 and C4; between C4 and C5; between C5 and C6; between C6 and C7; between C7 and T1; between T1 and T2; between T2 and T3; between T3 and T4; between T4 and T5; between T5 and T6; between T6 and T7; between T7 and T8; between T8 and T9; between T9 and T10; between T10 and T11; between T11 and T12; between T12 and L1; between L1 and L2; between L2 and L3; between L3 and L4; or between L4 and L5. In certain embodiments, the injury is to the cervical cord. In other embodiments, the injury is to the thoracic cord. In other embodiments the spinal cord injury is to the lumbrosacral cord. In certain other embodiments, the spinal cord injury is to the conus. In certain other embodiments, the CNS injury is to one or more nerves in the cauda equina. In another embodiment, the spinal cord injury is at the occiput.

In general, the dosage of administered antibodies will vary depending upon such factors as the patient's age, weight, height, sex, general medical condition and previous medical history. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be tested; each unit containing a predetermined quantity of active compound calculated to produce the desired therapeutic effect in association with the required pharmaceutical carrier. The specification for the dosage unit forms of the present invention are dictated by and directly dependent on (a) the unique characteristics of the active compound and the particular therapeutic or prophylactic effect to be achieved and (b) the limitations inherent in the art of compounding such an active compound for the treatment of sensitivity in individuals.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Administration of antibodies to a patient can be intravenous, intraarterial, intraperitoneal, intramuscular, subcutaneous, intrapleural, intrathecal, intraocular, intravitreal, by perfusion through a regional catheter, or by direct intralesional injection. When administering therapeutic proteins by injection, the administration may be by continuous infusion or by single or multiple boluses. Intravenous injection provides a useful mode of administration due to the thoroughness of the circulation in rapidly distributing antibodies. The antibody may be administered orally, for example, with an inert diluent or an assimilable edible carrier. The antibody and other ingredients, if desired, may be enclosed in a hard or soft shell gelatin capsule, compressed into tablets, buccal tablets, troches, capsules, elixirs, suspensions, syrups, wafers, and the like.

Anti-RGMa antibodies may be administered at low protein doses, such as 20 milligrams to 2 grams protein per dose, given once, or repeatedly, parenterally. Alternatively, the antibodies may be administered in doses of 20 to 1000 milligrams protein per dose, or 20 to 500 milligrams protein per dose, or 20 to 100 milligrams protein per dose.

Anti-RGMa antibodies may be administered at various times following spinal cord injury, including but not limited to less than 24 hours post spinal cord injury. In certain embodiments, an anti-RGMa antibody is administered to a subject less than 1, less than 2, less than 3, less than 4, less than 5, less than 6, less than 7, less than 8, less than 9, less than 10, less than 11, less than 12, less than 13, less than 14, less than 15, less than 16, less than 17, less than 18, less than 19, less than 20, less than 21, less than 22, or less than 23 hours post spinal cord injury. In certain specific embodiments, an anti-RGMa antibody is administered to a subject about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12 hours post spinal cord injury.

The anti-RGMa antibodies may be administered alone or they may be conjugated to liposomes, and can be formulated according to known methods to prepare pharmaceutically useful compositions, whereby the antibodies are combined in a mixture with a pharmaceutically acceptable carrier. A “pharmaceutically acceptable carrier” may be tolerated by a recipient patient. Sterile phosphate-buffered saline is one example of a pharmaceutically acceptable carrier. Other suitable carriers are well known to those in the art. See, for example, REMINGTON'S PHARMACEUTICAL SCIENCES, 19th Ed. (1995).

For purposes of therapy, antibodies are administered to a patient in a therapeutically effective amount in a pharmaceutically acceptable carrier. A “therapeutically effective amount” is one that is physiologically significant. The antibody is physiologically significant if its presence results in a detectable change in the physiology of a recipient patient. In the present context, the antibody may be physiologically significant if its presence results in, for example, decreased interferon-γ (INF-γ), interleukin-2 (IL-2), IL-4 and/or IL-17 secretion from CD4⁺ T cells. An agent is physiologically significant if its presence results in, for example, reduced proliferative responses and/or pro-inflammatory cytokine expression in peripheral blood mononuclear cells (PBMCs).

Additional treatment methods may be employed to control the duration of action of an antibody in a therapeutic application. Control release preparations can be prepared through the use of polymers to complex or adsorb the antibody. For example, biocompatible polymers include matrices of poly(ethylene-co-vinyl acetate) and matrices of a polyanhydride copolymer of a stearic acid dimer and sebacic acid. Sherwood et al., Bio/Technology 10:1446 (1992). The rate of release of an antibody from such a matrix depends upon the molecular weight of the protein, the amount of antibody within the matrix, and the size of dispersed particles. Saltzman et al., Biophys. J. 55:163 (1989); Sherwood et al., supra. Other solid dosage forms are described in REMINGTON'S PHARMACEUTICAL SCIENCES, 19th ed. (1995).

(1) Neurologic Recovery

Neurologic recovery can be assessed using available measures, including, but not limited to Frankel grade, motor score, and the American Spinal Injury Association (ASIA) Impairment Scale (AIS). The AIS a clinical tool to assess motor and sensory neurologic intactness.

In some embodiments, an improvement in one or more symptoms of, or a reduction in the progression of one or more symptoms of, SCI is assessed in accordance with the International Standards for Neurological and Functional Classification of Spinal Cord Injury. The International Standards for Neurological and Functional Classification of Spinal Cord Injury, published by ASIA, is a widely accepted system describing the level and extent of SCI based on a systematic motor and sensory examination of neurologic function. See International Standards For Neurological Classification Of Spinal Cord Injury, J Spinal Cord Med. 34(6):535-46 (2011), the disclosure of which is hereby incorporated by reference in its entirety.

(2) Functional Recovery

Functional recovery may be achieved in conjunction with or independently of neurologic recovery. Functional recovery can be assessed using available measures, including, but not limited to the Spinal Cord Independence Measure (SCIM), the Functional Independence Measure (FIM), the Walking Index for Spinal Cord Injury (WISCI), the Modified Barthel Index (MBI), the Quadriplegia Index of Function (QIF), the London Handicap scale, and Short Form 36. See, e.g., Anderson K. et al. Functional Recovery Measures for Spinal Cord Injury: An Evidence-Based Review for Clinical Practice and Research. J. Spinal Cord Med. 31, 133-144 (2008). In other embodiments, functional recovery may be assessed using the open field Basso, Beattie and Bresnahan (BBB) locomotor test, gait analysis, ladderwalk analysis, and/or the tests that form the combined behavioral score (CBS).

b. Pain

In any subject, an assessment may be made as to whether the subject has, or is at risk of experiencing, any type of acute or chronic pain condition or disorder, including nociceptive pain, neuropathic pain or a combination thereof. Such pain conditions or disorders can include, but are not limited to, post-operative pain, osteoarthritis pain, pain due to inflammation, rheumatoid arthritis pain, musculoskeletal pain, burn pain (including sunburn), ocular pain, the pain associated with dental conditions (such as dental caries and gingivitis), post-partum pain, bone fracture, herpes, HIV, traumatic nerve injury, stroke, post-ischemia, fibromyalgia, reflex sympathetic dystrophy, complex regional pain syndrome, spinal cord injury, sciatica, phantom limb pain, diabetic neuropathy, hyperalgesia and cancer. The assessment may indicate an appropriate course of therapy, such as preventative therapy, maintenance therapy, or modulative therapy. Accordingly, provided herein is a method of treating, preventing, modulating, or attenuating a spinal cord injury by administering a therapeutically effective amount of one or more of the antibodies described herein (such as, for example, antibody AE12-1, AE12-1-Y, or AE12-1-Y-QL). The antibody may be administered to a subject in need thereof. The antibody may be administered in a therapeutically effective amount.

In general, the dosage of administered antibodies will vary depending upon such factors as the patient's age, weight, height, sex, general medical condition and previous medical history. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be tested; each unit containing a predetermined quantity of active compound calculated to produce the desired therapeutic effect in association with the required pharmaceutical carrier. The specification for the dosage unit forms of the present invention are dictated by and directly dependent on (a) the unique characteristics of the active compound and the particular therapeutic or prophylactic effect to be achieved and (b) the limitations inherent in the art of compounding such an active compound for the treatment of sensitivity in individuals.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Administration of antibodies to a patient can be intravenous, intraarterial, intraperitoneal, intramuscular, subcutaneous, intrapleural, intrathecal, intraocular, intravitreal, by perfusion through a regional catheter, or by direct intralesional injection. When administering therapeutic proteins by injection, the administration may be by continuous infusion or by single or multiple boluses. Intravenous injection provides a useful mode of administration due to the thoroughness of the circulation in rapidly distributing antibodies. The antibody may be administered orally, for example, with an inert diluent or an assimilable edible carrier. The antibody and other ingredients, if desired, may be enclosed in a hard or soft shell gelatin capsule, compressed into tablets, buccal tablets, troches, capsules, elixirs, suspensions, syrups, wafers, and the like.

Anti-RGMa antibodies may be administered at low protein doses, such as 20 milligrams to 2 grams protein per dose, given once, or repeatedly, parenterally. Alternatively, the antibodies may be administered in doses of 20 to 1000 milligrams protein per dose, or 20 to 500 milligrams protein per dose, or 20 to 100 milligrams protein per dose.

Anti-RGMa antibodies may be administered at various times following a spinal cord injury where there is a risk of developing neuropathic pain, including but not limited to less than 24 hours post spinal cord injury. In certain embodiments, an anti-RGMa antibody is administered to a subject less than 1, less than 2, less than 3, less than 4, less than 5, less than 6, less than 7, less than 8, less than 9, less than 10, less than 11, less than 12, less than 13, less than 14, less than 15, less than 16, less than 17, less than 18, less than 19, less than 20, less than 21, less than 22, or less than 23 hours post spinal cord injury. In certain specific embodiments, an anti-RGMa antibody is administered to a subject about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, or about 12 hours post spinal cord injury.

The antibodies may be administered alone or they may be conjugated to liposomes, and can be formulated according to known methods to prepare pharmaceutically useful compositions, whereby the antibodies are combined in a mixture with a pharmaceutically acceptable carrier. A “pharmaceutically acceptable carrier” may be tolerated by a recipient patient. Sterile phosphate-buffered saline is one example of a pharmaceutically acceptable carrier. Other suitable carriers are well known to those in the art. See, for example, REMINGTON'S PHARMACEUTICAL SCIENCES, 19th Ed. (1995).

For purposes of therapy, antibodies are administered to a patient in a therapeutically effective amount in a pharmaceutically acceptable carrier. A “therapeutically effective amount” is one that is physiologically significant. The antibody is physiologically significant if its presence results in a detectable change in the physiology of a recipient patient. In the present context, the antibody may be physiologically significant if its presence results in, for example, decreased interferon-γ (INF-γ), interleukin-2 (IL-2), IL-4 and/or IL-17 secretion from CD4+ T cells. An agent is physiologically significant if its presence results in, for example, reduced proliferative responses and/or pro-inflammatory cytokine expression in peripheral blood mononuclear cells (PBMCs).

Additional treatment methods may be employed to control the duration of action of an antibody in a therapeutic application. Control release preparations can be prepared through the use of polymers to complex or adsorb the antibody. For example, biocompatible polymers include matrices of poly(ethylene-co-vinyl acetate) and matrices of a polyanhydride copolymer of a stearic acid dimer and sebacic acid. Sherwood et al., Bio/Technology 10:1446 (1992). The rate of release of an antibody from such a matrix depends upon the molecular weight of the protein, the amount of antibody within the matrix, and the size of dispersed particles. Saltzman et al., Biophys. J. 55:163 (1989); Sherwood et al., supra. Other solid dosage forms are described in REMINGTON'S PHARMACEUTICAL SCIENCES, 19th ed. (1995).

(1) Neuropathic Pain

As used herein the term “neuropathic pain” refers to pain that results from injury to a nerve, spinal cord, or brain, and often involves neural supersensitivity. Examples of neuropathic pain include chronic lower back pain, pain associated with arthritis, cancer-associated pain, herpes neuralgia, phantom limb pain, central pain, opioid resistant neuropathic pain, bone injury pain, and pain during labor and delivery. Other examples of neuropathic pain include post-operative pain, cluster headaches, dental pain, surgical pain, pain resulting from severe, for example third degree, burns, post partum pain, angina pain, genitourinary tract related pain, and including cystitis.

Neuropathic pain can be distinguished from nociceptive pain. Pain involving a nociceptive mechanism usually is limited in duration to the period of tissue repair and generally is alleviated by available analgesic agents or opioids (Myers (1995) Regional Anesthesia 20:173). Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Unlike nociceptive pain, neuropathic pain generally is resistant to opioid therapy (Myers, supra, 1995). Accordingly, antibodies disclosed herein can be used to treat neuropathic pain.

As used herein the term “nociceptive pain” refers to pain that is transmitted across intact neuronal pathways, i.e., pain caused by injury to the body. Nociceptive pain includes somatic sensation and normal function of pain, and informs the subject of impending tissue damage. The nociceptive pathway exists for protection of the subject, e.g., the pain experienced in response to a burn). Nociceptive pain includes bone pain, visceral pain, and pain associated with soft tissue.

### 5. EXAMPLES

The present invention has multiple aspects, illustrated by the following non-limiting examples.

### General Methods for Example 1

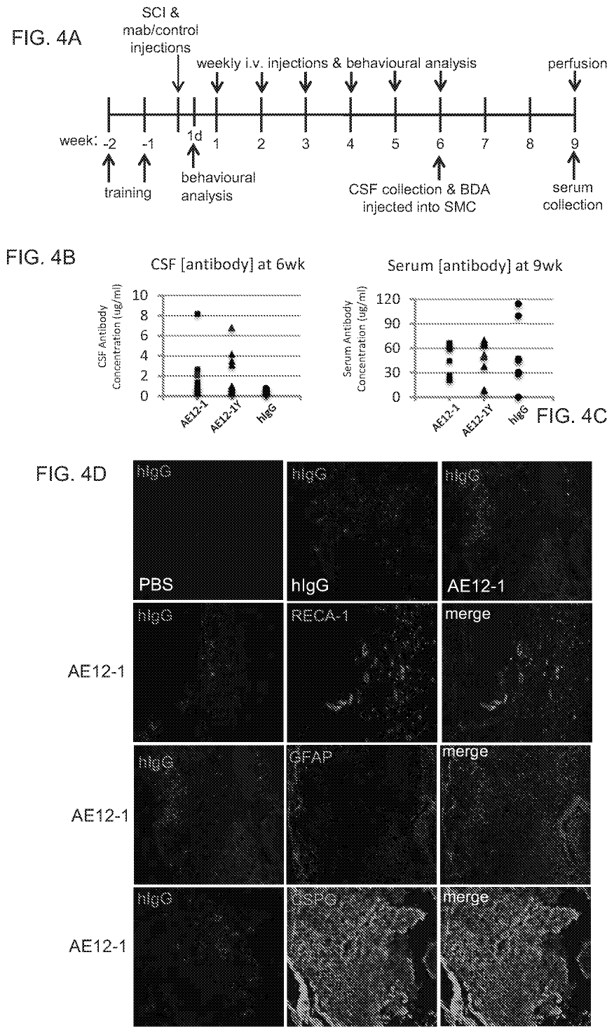

A summary of the study design is depicted in FIG. 4A. Adult female Wistar rats were pre-trained and then clip impact-compression SCI was made at T8 with a 20 g force for 1 min with a modified aneurysm clip according to published protocols. Briefly, the clip was held open with a clip applicator, with the lower blade of the clip passed extradurally and ventrally around the spinal cord. The clip was then rapidly released from the applicator to produce a bilateral impact force followed by sustained dorsal-ventral compression. This is a clinically relevant model of SCI reflecting human pathology. The combination of acute impact followed by persisting compression is the most common mechanism of SCI in humans; the acute clip compression model can simulate this impact-compression injury.

Clip impact-compression was immediately followed by local intraspinal and systemic intravenous injections (20 mg/kg) of either AE12-1, AE12-1-Y, hIgG isotype control, or PBS vehicle, once per week until 6 weeks post-SCI.

### Example 1.1

### RGMa Expression in Rat and Human Spinal Cord after SCI

Methodology: Rat tissue sections were prepared for immunohistochemical staining and incubated with primary antibodies overnight at 4° C. The following primary antibodies were used: NeuN (1:500, Millipore Bioscience Research Reagents) for neurons, GFAP (1:200, Millipore) for astrocytes, Iba-1 (1:1000, Wako Chemicals) for activated macrophages/microglia, CS56 (1:500, Sigma) for chondroitin sulfate proteoglycan, calcitonin gene-related peptide (CGRP) (1:1000, Millipore) for sensory fibers, 5HT (1:3000, ImmunoStar) for serotonergic fibers, and hIgG (1:500, Millipore) to detect human IgG antibodies. Sections were incubated overnight with primary antibody diluted in blocking solution, washed, and incubated with fluorescent-conjugated secondary antibody.

Human tissue sections were prepared for staining and incubated overnight with primary antibody (1:200 RGMa, Abcam; or 1:100 Neogenin, Santa Cruz) diluted in blocking solution. Sections were washed, incubated with biotinylated anti-mouse secondary antibody (1:500, Vector Laboratories), washed, and incubated with avidin-biotin-peroxidase complex (Vectastain Elite ABC Kit Standard, Vector Laboratories). Diaminobenzidine (DAB) (Vectastain Elite ABC Kit Standard, Vector Laboratories) was applied as the chromogen.

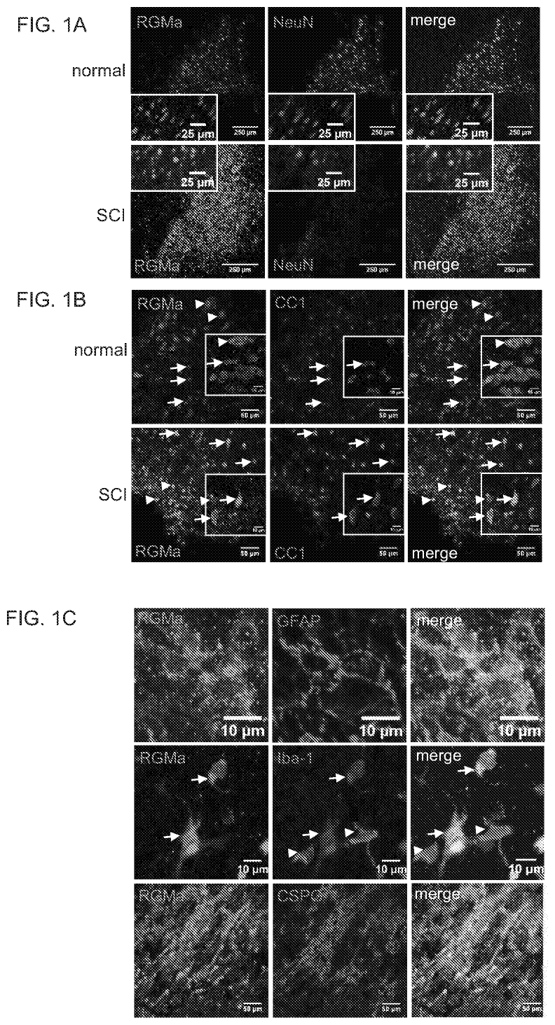

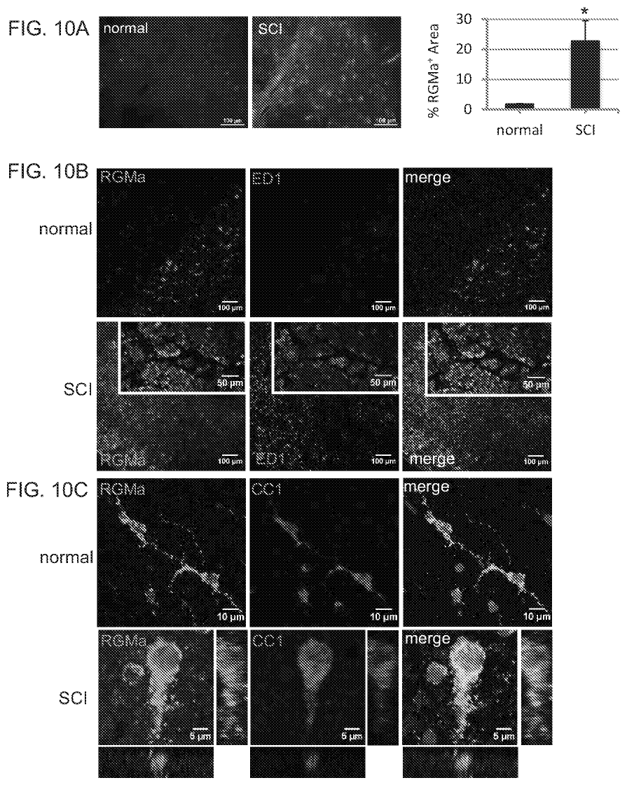

Results: RGMa was upregulated after clip impact-compression injury of the rat spinal cord (FIG. 1, FIG. 10). As shown by double-label immunostaining, RGMa was primarily expressed by neurons (RGMa+/NeuN+) and oligodendrocytes (RGMa+/CC1+) in the normal rat spinal cord (FIGS. 1A and B). Quantification at 1 week post-injury showed a 15-fold increase in RGMa expression (FIG. 10A). After SCI, RGMa was expressed in neurons (FIG. 1A), oligodendrocytes (FIG. 1B, FIG. 10C), astrocytes as shown by GFAP labeling (FIG. 1C), activated microglia and macrophages as shown by Iba-1 (FIG. 1C) and ED-1 (FIG. 10B), and within CSPG scar-rich regions within and surrounding the lesion site (FIG. 1C).

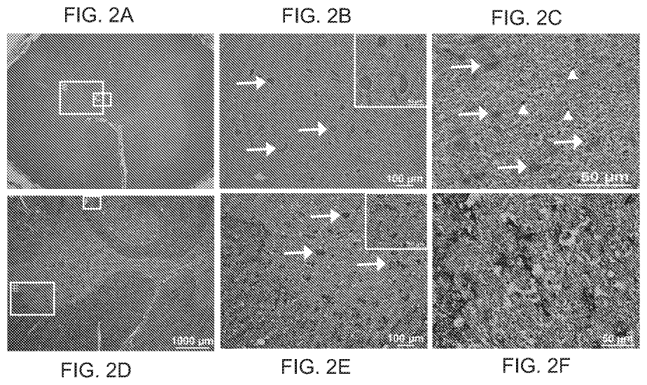

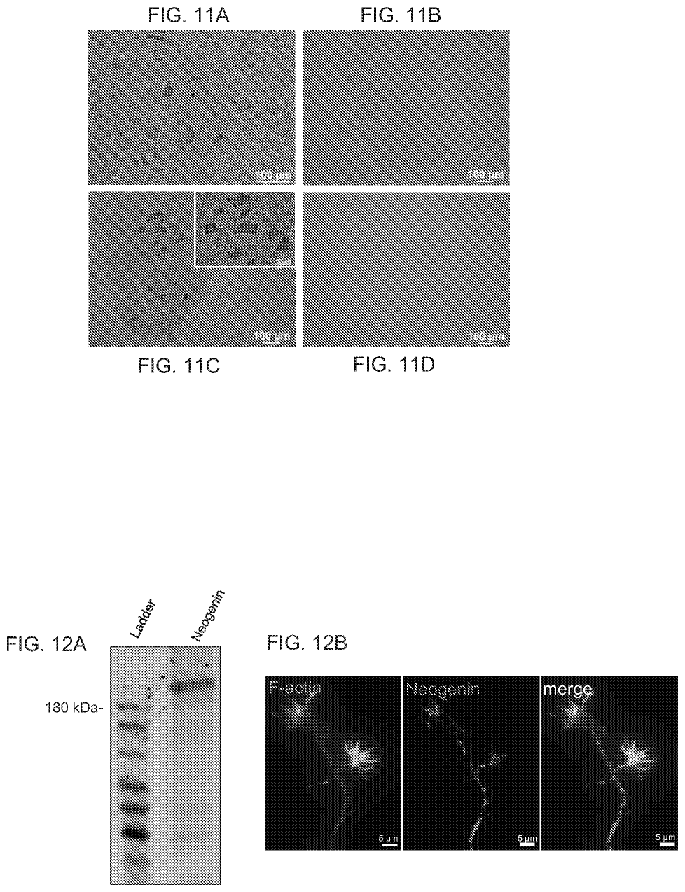

In the uninjured human spinal cord, RGMa was expressed at low levels in neurons, as shown by immunostaining of neurons in the intermediate gray matter (FIGS. 2A and B) and oligodendrocytes in the dorsal column (FIG. 2C). In the injured human spinal cord at 3 days post-SCI, RGMa expression was upregulated in neurons, axons, oligodendrocytes, and myelin enriched white matter regions (FIG. 2D-F). Furthermore, the RGMa receptor Neogenin, was expressed by neurons in both rat (data not shown) and human spinal cord, and was also upregulated after injury (FIG. 11).

### Example 1.2

### Effect of an Anti-RGMa Antibody on Neurite Outgrowth In Vitro

Methodology: For the neurite outgrowth assay, E18 mouse cortical neurons were plated on poly-L-Lysine coated glass cover slips treated with laminin (Invitrogen; 10 mg/ml) and RGMa proteins (5 mg/ml) and incubated for 24 hours at 37° C. with control antibody (hIgG) or anti-RGMa (1 mg/ml). For the neurite outgrowth analysis, cortical cells were immunostained with βIII-tubulin (Sigma; 1:500). For western blots, mouse cortical neurons were lysed in RIPA buffer and loaded on a 10% acrylamide gel before transfer onto a nitro-cellulose membrane. Blots were probed with the anti-RGMa antibodies (AE12-1 and AE12-1-Y) and anti-Neogenin (E20; Santa Cruz; 10 mg/ml).

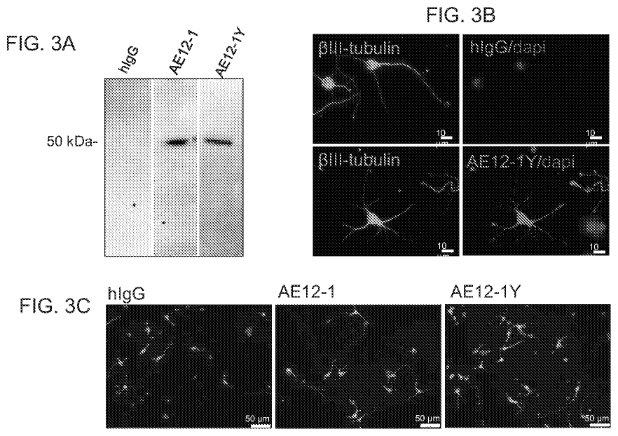

Results: In western blots of mouse cortical neuron lysates, both AE12-1 and AE12-1-Y specifically detected a 50-kDa band (FIG. 3A). Cultured mouse primary cortical neurons were also shown to express RGMa, as shown by immunostaining with AE12-1-Y (FIG. 3B, AE12-1 immunostaining not shown). The anti-RGMa antibodies promote neurite outgrowth in vitro. Cultured embryonic mouse cortical neurons plated on laminin and inhibitory RGMa showed minimal extension of neurites when incubated with hIgG whereas incubation with AE12-1 and AE12-1-Y RGMa antibodies resulted in more extensive neurite outgrowth compared to cells plated on laminin alone. In western blots, the Neogenin receptor was detected as a 200-kDa band and Neogenin was also expressed by cultured mouse cortical neurons (FIG. 12).

### Example 1.3

### Detection of an Anti-RGMa Antibody Serum, CSF, and Spinal Cord

Methodology: At 6 weeks post-SCI, cerebrospinal fluid (CSF) was sampled via lumbar puncture (LP). At 9 weeks post-SCI, 3 weeks after the last antibody dose, serum was collected and rats were perfused. Using an ELISA assay, the concentration of antibody in CSF and serum samples from AE12-1, AE12-1-Y, and hIgG treated rats was determined.

Results: The antibody concentration in the CSF of rats injected with AE12-1 ranged from 0.25-8.20 μg/ml, and for AE12-1-Y the antibody concentration range was 0.33-6.77 μg/ml (FIG. 4B). In comparison, antibody concentration in serum was considerably higher, approximately 10-fold greater than in CSF as the antibodies were injected intravenously after the initial intraspinal injections immediately following SCI (FIG. 4C). Furthermore, antibody concentration remained elevated in serum 3 weeks after the last dose. The human antibodies were detected in the injured rat spinal cord by immunostaining of rat spinal cord tissue with anti-human IgG. Human IgG immunoreactivity was detected in tissue from rats injected with AE12-1 (or AE12-1-Y, not shown in figure) or hIgG control antibody but not in PBS vehicle controls (FIG. 4D). Staining of human IgG was apparent around blood vessels and within CSPG positive scar tissue around the lesion site (FIG. 4D). There was no difference in staining in tissue injected with either AE12-1 or AE12-1-Y or hIgG.

### Example 1.4

### Anti-RGMa Antibody Promotes Functional Recovery

Methodology: Functional tests were performed before the injury for pre-training and to obtain baseline assessment scores, again at 1 day after SCI, and then weekly for 6 weeks. Neurological recovery was monitored weekly using the BBB locomotor rating scale, motor subscore, and horizontal ladderwalk test.

Locomotor function was evaluated using the Basso, Beattie and Bresnahan (BBB) locomotor rating scale, which ranges from 0 (no hind limb movement) to 21 (normal locomotion). Motor subscores were used to assess additional measures such as toe clearance, predominant paw position, and absence of instability.

Ladderwalk analysis was used to assess fine motor function. Weekly post-SCI, rats with a BBB score >11 were placed on the horizontal ladderwalk apparatus and 3 runs were recorded. Recordings were analyzed in slow motion, and the total number of footfalls per hindlimb was scored for each run and averaged. Injured rats with dragging hindlimbs were scored the maximum footfalls which was 6 footfalls per hindlimb. Uninjured rats had 0 or occasionally 1 footfall per crossing.

To further elucidate motor function, gait analysis was performed using the CatWalk system (Noldus Information Technology, Wageningen, Netherlands). Baseline computerized gait assessments were obtained pre-operatively and compared to assessment at 6 weeks post-SCI. The CatWalk analysis system has been described in detail elsewhere. See Hamers F P, Lankhorst A J, van Laar T J, Veldhuis W B, and Gispen W H. Automated quantitative gait analysis during overground locomotion in the rat: its application to spinal cord contusion and transection injuries. J Neurotrauma. 2001; 18(2):187-201.

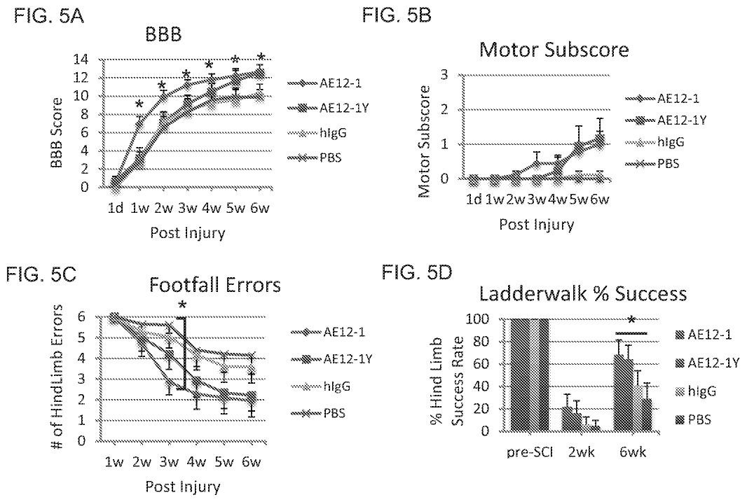

Results: Acute treatment with AE12-1 showed significant recovery of the BBB as early as 1 week post-SCI compared to PBS or hIgG controls which was maintained for the duration of the trial (FIG. 5A). In contrast, AE12-1-Y showed a delayed improvement on the BBB with a statistically significant difference at 6 weeks post-SCI relative to controls (12.6 vs 9.9 PBS) (FIG. 5A). The difference in recovery profiles of AE12-1 and AE12-1-Y may be due to the longer half-life of AE12-1. Both AE12-1 and AE12-1-Y also showed a trend towards a higher motor subscore compared to controls (FIG. 5B).

In the ladderwalk test, footfall errors are scored and, thus, a higher score reflects poorer coordination. The ladderwalk test showed a trend toward reduced footfall errors in rats treated with AE12-1 or AE12-1-Y, with a statistically significant difference at 3 weeks post-SCI for AE12-1 (p<0.05) and approaching significance at weeks 4, 5, and 6 (p=0.067, p=0.089, p=0.07) (FIG. 5C). At 6 weeks post-SCI, both AE12-1 and AE12-1-Y treated rats showed a higher percentage of successful hindlimb steps (68.4% and 64.2%) compared to controls (hIgG 41%, PBS 29%) (FIG. 5D).

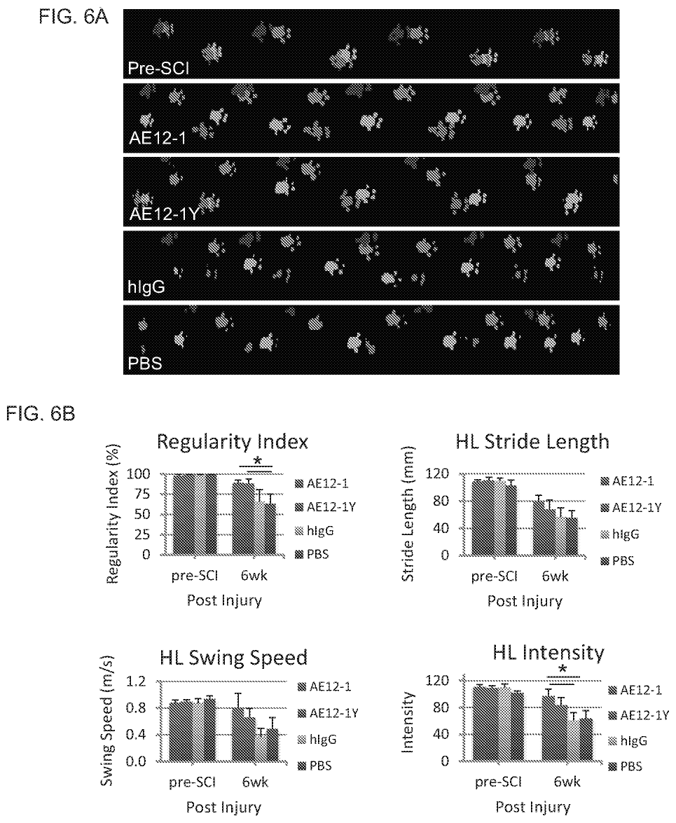

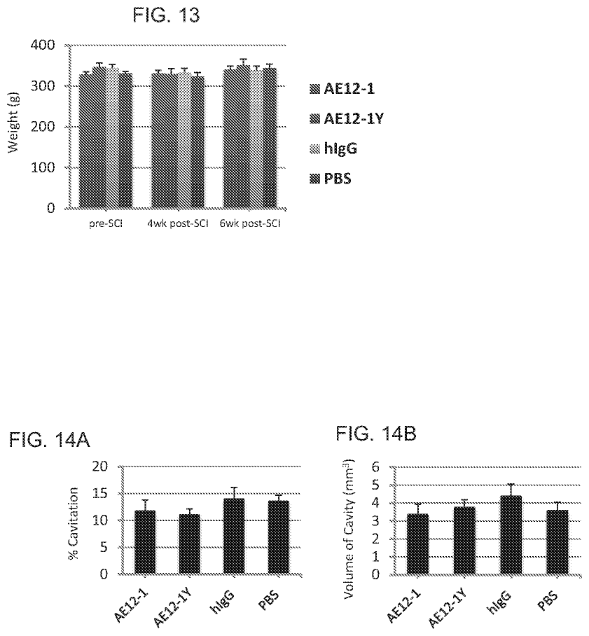

To further characterize the effects of RGMa neutralization on neurobehavioral function, gait analysis was performed using the CatWalk system at 6 weeks post-SCI and treatment (FIG. 6). Both AE12-1 and AE12-1-Y treated rats showed significant improvement in the regularity index relative to control groups, reflecting better inter-paw coordination (AE12-1: 89.3%; AE12-1-Y: 88.3%; hIgG: 65.8%; PBS: 63.4%) (FIG. 6). The AE12-1 and AE12-1-Y treated rats also showed a trend towards improved hindlimb stride length and swing speed approaching the pre-SCI values, although this did not reach statistical significance. Interestingly, the mean intensity with which the hindpaws contacted the glass walkway significantly increased in AE12-1 treated rats compared to controls (FIG. 6). In addition, treatment with AE12-1 or AE12-1-Y did not alter rat weight (FIG. 13).

### Example 1.5

### Anti-RGMa Antibody Promotes Neuronal Survival

Methodology: At 9 weeks post-SCI (i.e., after 6 weeks of AE12-1 or AE12-1-Y treatment), perilesional neurons were quantified with the neuronal marker NeuN.

A separate experiment was performed where two groups of rats were injured and injected (both intraspinally and intravenously exactly as described above) with either AE12-1 or PBS. These rats were sacrificed at 7 hours post-SCI/injections. Double-labeling with NeuN and TUNEL staining was performed.

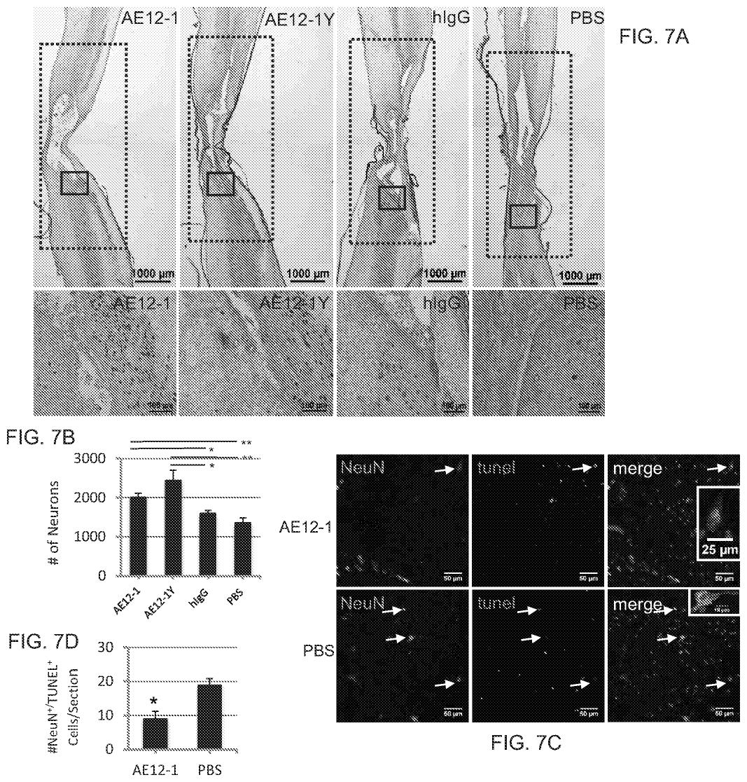

Results: Six weeks of treatment with the RGMa antibodies increased the number of perilesional neurons approximately 1.5-fold compared to controls (FIGS. 7A and B).

To determine if the neuronal sparing was due to fewer neurons undergoing apoptosis after injury, neuronal cell death was assessed at the 7 hour post-SCI/injection time point. There were significantly fewer NeuN/TUNEL positive neurons in AE12-1 treated rats relative to control (2-fold difference) (FIGS. 7C and D). There was no significant difference between groups in either the percentage cavitation or the volume of the cavity (FIGS. 14A and 14B).

### Example 1.6

### RGMa Antibodies Reduce Astrogliosis and CSPG Expression

Methodology: The % GFAP positive area in regions rostral and caudal to the lesion site was quantified. For quantification of GFAP immunoreactivity, 3 consecutive serial sections (160 μm apart) in each cord containing the maximal area of cavitation were used for quantification. CSPG immunoreactivity was similarly quantified.

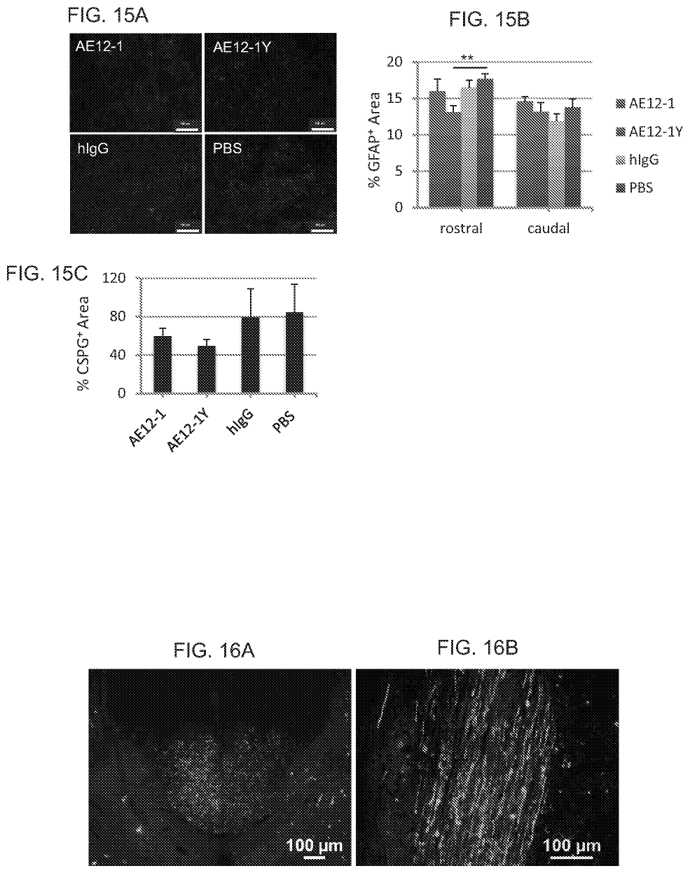

Results: A significant reduction in astrogliosis rostral to the lesion was observed in AE12-1-Y treated rats (FIGS. 15A and 15B). There was a trend towards reduced CSPG expression around the lesion site in AE12-1 and AE12-1-Y treated rats (FIG. 15C).

### Example 1.7

### Anti-RGMa Antibody Promotes Axonal Regeneration

Methodology: To visualize axons from the corticospinal tract (CST), anterograde axonal tracing with biotinylated dextran amines (BDA) was performed 6 weeks after SCI following completion of the functional assessment. BDA was injected into the sensorimotor cortex (SMC) to anterogradely label the CST. The SCI model of impact-compression injury in which both the dorsal and ventral aspects of the spinal cord are simultaneously compressed results in central cavitation of the gray matter and adjacent white matter, severing all CST axons in the dorsal CST, leaving only a spared rim of subpial white matter. The intensity of BDA staining of the dorsal CST in the rostral segment of each cord was quantified and the ratio was used to normalize the counts of BDA labeled fibers to correct for inter-animal variation in the BDA labeling efficiency. The caudal segment was examined for the presence of any BDA labeled fibers.

In a separate experiment, rats were injured and injected with AE12-1-Y as described above, and then injected with BDA at 4 weeks or at 6 weeks post-SCI. Rats were sacrificed 3 weeks after injection and the number of BDA labeled axons and their length was quantitated as described above.

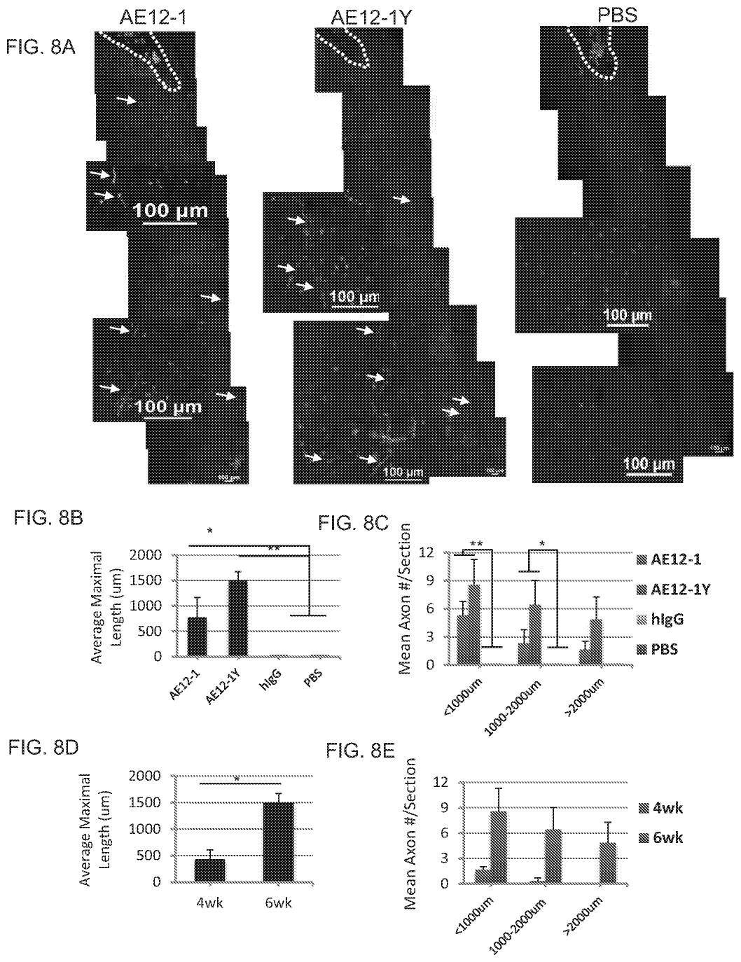

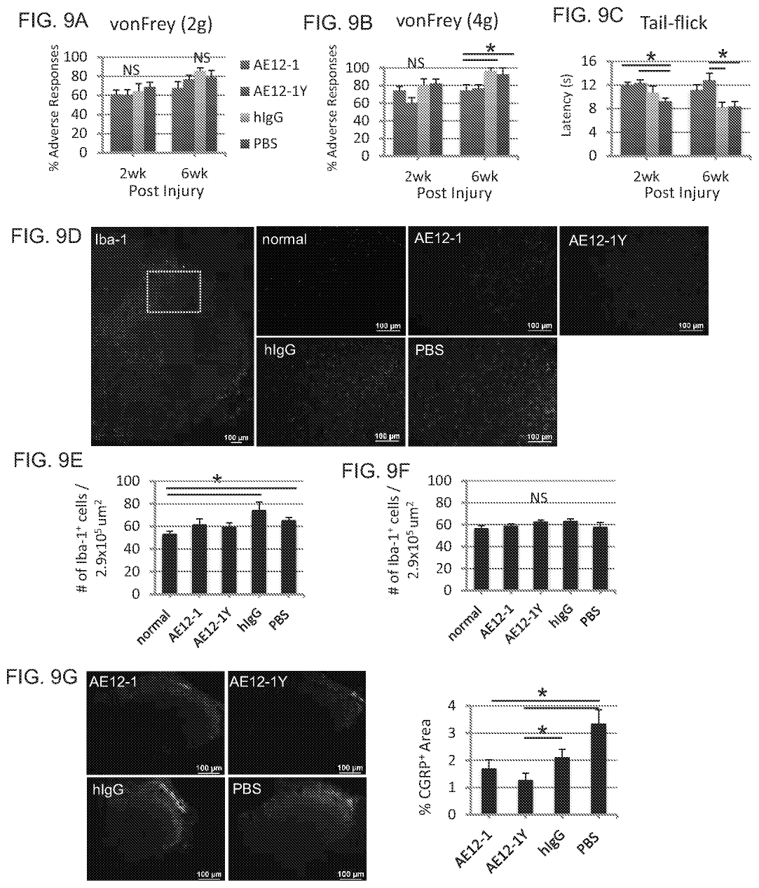

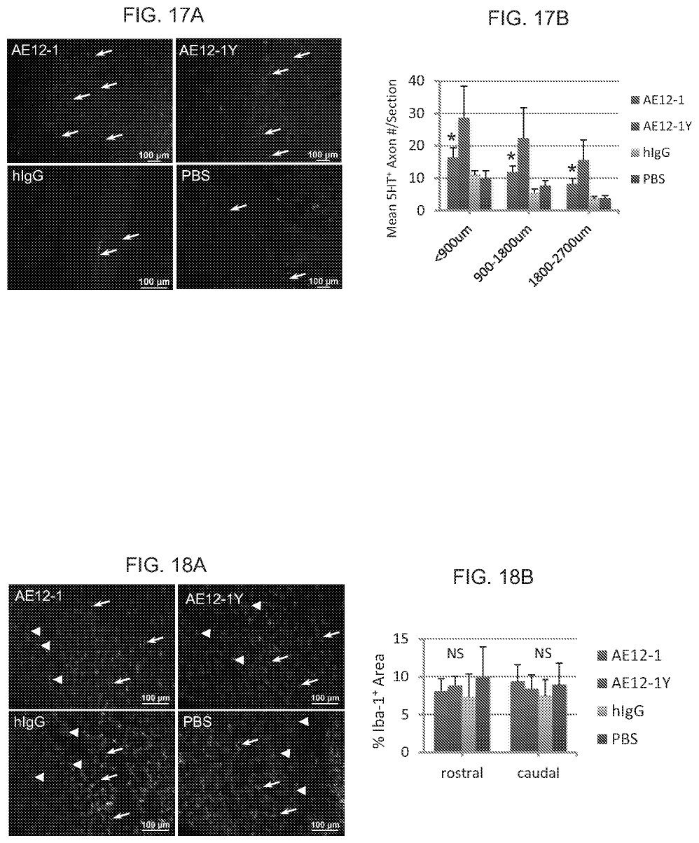

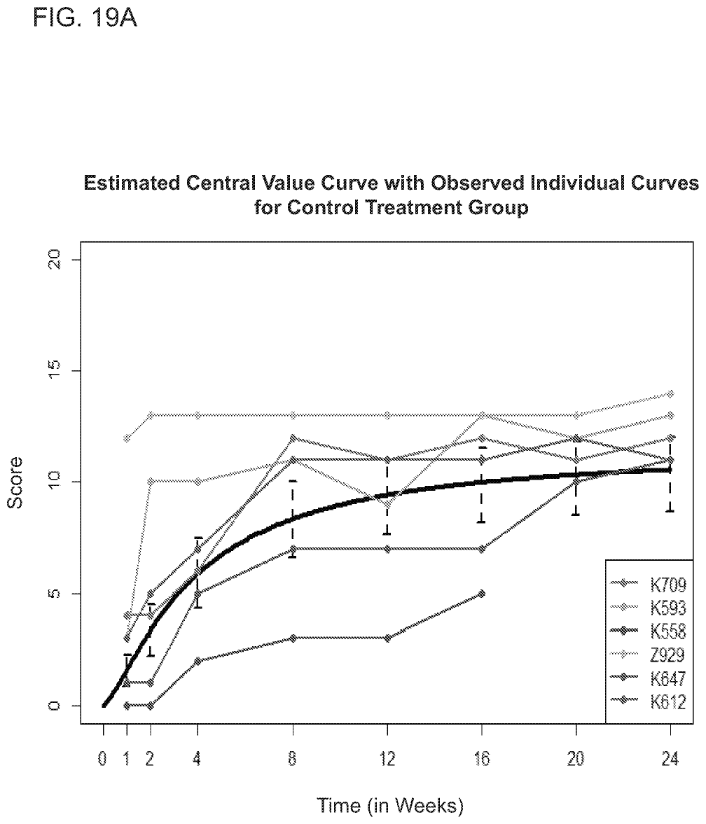

Results: Treatment with AE12-1 or AE12-1-Y showed BDA labeled CST fibers caudal to the lesion site (FIG. 8A). These fibers showed a highly irregular morphology, unlike BDA labeled CST fibers rostral to the lesion or in uninjured rats which are typically long and straight (FIG. 16). Both the number and average maximal length of BDA labeled CST fibers was increased in rats injected with either AE12-1 or AE12-1-Y (FIGS. 8C and D). In contrast, no BDA fibers were observed caudal to the lesion site in control rats. There were more BDA labeled regenerated CST fibers at 6 weeks compared to 4 weeks and BDA labeled fibers were significantly longer at 6 weeks (FIGS. 8E and F) indicating the regeneration of CST axons following treatment with either AE12-1 or AE12-1-Y. In an analysis of descending serotonergic pathways, a significantly higher number of 5HT+ serotonergic fibers caudal to the lesion site were observed in AE12-1 treated rats relative to controls (FIG. 17). Rats injected with AE12-1-Y showed a trend towards a higher number of 5HT labeled axons although there was significant variability in the number of 5HT fiber counts in the AE12-1-Y groups as reflected in the large standard error (FIG. 17).

### Example 1.8

### Anti-RGMa Antibody Attenuates Neuropathic Pain

Methodology: For tests for neuropathic pain, mechanical allodynia was assessed with vonFrey filaments and the tail flick test was used for thermal hyperalgesia. All tests were performed by 2 independent examiners blinded to treatments.

VonFrey filaments (Stoelting) were used to assess mechanical allodynia at 2 and 6 weeks post-SCI. The filaments were used to assess cutaneous sensitivity to normally innocuous mechanical stimulation and were applied to the dermatomes as described by Takahashi (2003) to determine mechanical allodynia at the level of the SCI. A 2 g and 4 g filament was used at each time point.

Thermal hyperalgesia was evaluated by the tail flick test by recording the latency to withdrawal of the tail in response to noxious skin heating. An automated analgesia meter (IITC Life Science, Woodland Hills, Calif.) was used to apply a beam of light to the dorsal surface of the tail at 4 cm from the tip.

Results: Interestingly, treatment with either AE12-1 or AE12-1-Y reduced at-level mechanical allodynia and thermal hyperalgesia. At 6 weeks post-SCI, rats administered AE12-1 showed significantly fewer adverse responses to the 4 g vonFrey stimulus compared to controls (FIGS. 9A and 9B). Rats treated with either AE12-1 or AE12-1-Y showed reduced latency to withdrawal of the tail to a heat stimulus compared to controls (FIG. 9C). No significant difference in the percentage area of Iba-1+ staining rostral or caudal to the lesion site was found (FIG. 18). The quantitation included all Iba-1 immunostained cells, activated microglia and macrophages as shown in FIG. 18A, thus this quantification reflected both cell types. However, Iba-1+ macrophages can easily be distinguished morphologically rostral or caudal to the lesion site, thus activated microglia caudal to the lesion could be specifically quantified. In this analysis, only Iba-1+ microglia were counted (FIG. 9D). Although not statistically significant, there were fewer Iba-1+ microglia in the dorsal horn at T10 in AE12-1 or AE12-1-Y treated rats relative to controls. Conversely, significantly more Iba-1+ cells were counted in the dorsal horn in injured controls compared to normal cord (FIG. 9E). There was no significant difference between groups for Iba-1+ microglia in the dorsal horn of spinal cords at C4 (FIG. 9F), highlighting the differences seen caudal to the lesion at T10 (FIG. 9E). Furthermore, control rats showed significantly greater CGRP+ immunoreactive fibers in the dorsal horn compared to AE12-1 or AE12-1-Y treated rats (FIG. 9G), suggesting a positive effect of RGMa neutralization on the plasticity of pain afferents entering the dorsal horn caudal to the level of injury.

### Example 2

Adult male African Green Monkeys were randomized into three treatment groups (n=8/group). Each animal was implanted with a vascular access port (VAP) and microinfusion pump (Azlet). Following VAP/pump implantation, animals were subjected to clip hemicompression SCI at T9/10 with a 760 g force for 5 or 30 min, as generally described above.

Animals in the intravenous group were treated with AE12-1-Y-QL (25 mg/kg) beginning 75 minutes after clip impact-compression. Treatment with AE12-1-Y-QL continued once every two weeks for 24 weeks post-SCI. Animals in the intravenous group additionally received a control IgG antibody by continuous intrathecal infusion.

For animals in the intrathecal group, microinfusion pumps were activated at the time of implant with an initial elution time of 4 hours post SCI. AE12-1-Y-QL (150 μg/kg/day) was continuously infused for four months. Animals in the intrathecal group additionally received a control IgG antibody intravenously as described above.

Animals in the control group received the control IgG antibody intravenously as well as by continuous intrathecal infusion.

Functional assessment was performed prior to surgery and at 1, 2, 4, 8, 12, 16, 20, and 24 weeks post SCI. Neuromotor scores were obtained as described previously. Briefly, the rating scale ranges from 0 to 20 as shown in Table 4.

TABLE 4

| Score | Description |
|---|---|
| 0 | No voluntary function |
| 1 | Slight one or two joints movement |
| 2 | Active one or two joints, slight movement others |
| 3 | Active movement of all three joints, no weight bearing |
| 4 | Slight weight bearing, consistent dorsal stepping (no plantar |
| | stepping) |
| 5 | Slight weight bearing, occasional plantar stepping |
| 6 | Frequent plantar stepping, occasional weight bearing, hops with |
| | partial weight support |
| 7 | Frequent plantar stepping and weight bearing, occasional arm-leg |
| | coordination |
| 8 | Consistent plantar stepping and partial weight supported steps, |
| | occasional arm-leg coordination |
| 9 | Frequent partial weight supported steps, occasional arm-leg |
| | coordination |
| 10 | Occasional partial weight supported steps, frequent foot drop |
| | and/or drag, run with partial weight support |
| 11 | Occasional partial weight supported steps, frequent arm-leg |
| | coordination |
| 12 | Slight partial weight supported steps, frequent arm-leg |
| | coordination, stands up on leg with partial weight support |
| 13 | Slight partial weight supported steps, consistent arm-leg |
| | coordination, frequent foot drop and/or drag |
| 14 | Full weight supported steps and consistent arm-leg coordination, |
| | occasional foot drop and/or drag |
| 15 | Occasional foot drop and/or drag, stand up on leg with full weight |
| | support |
| 16 | Slight foot drop and/or drag, no toe clearance |
| 17 | No foot drop and/or drag, no toe clearance |
| 18 | Occasional toe clearance |
| 19 | Frequent toe clearance |
| 20 | Normal |

Neuromotor scores were obtained using video tapes exemplifying each behavior being scored. Scorers were tested with control videos every 3 months to confirm consistency.

Eₘₐₓ is defined as the maximum neuromotor score that a monkey will be able to reach. ET₅₀ is defined as the time when monkeys reach half of Eₘₐₓ (i.e., the maximum level of recovery). Data are presented in Table 5 and Table 6 below. Six animals from the control group and seven animals from each treatment group were included in the analysis.

TABLE 5
Statistical Analysis for Eₘₐₓ Model

| | | | 95% Confidence |
|---|---|---|---|
| Parameter | Estimate | Standard Error | Interval |
| Eₘₐₓ (Ctrl) | 11.44 | 0.98 | (9.39, 13.49) |
| Eₘₐₓ (IV) | 14.03 | 0.97 | (11.99, 16.07) |
| Eₘₐₓ (IT) | 11.69 | 0.96 | (9.69, 13.70) |
| ET₅₀ | 3.84 | 0.66 | (2.46, 5.22) |

TABLE 6
Estimated Differences of Emax between Treatment Groups

| | | | | 95% Confidence |
|---|---|---|---|---|
| Parameter | Estimate | Std. Err. | P-value# | Interval |
| Eₘₐₓ (IV) − | 2.59 | 1.24 | 0.026 | (−0.017, 5.201) |
| Eₘₐₓ (Ctrl) | | | | |
| Eₘₐₓ (IT) − | 0.25 | 1.25 | 0.421 | (−2.370, 2.877) |
| Eₘₐₓ (Ctrl) | | | | |
| Eₘₐₓ (IV) − | 1.95 | 1.20 | 0.067 | (−0.176, 4.854) |
| Eₘₐₓ (IT) | | | | |

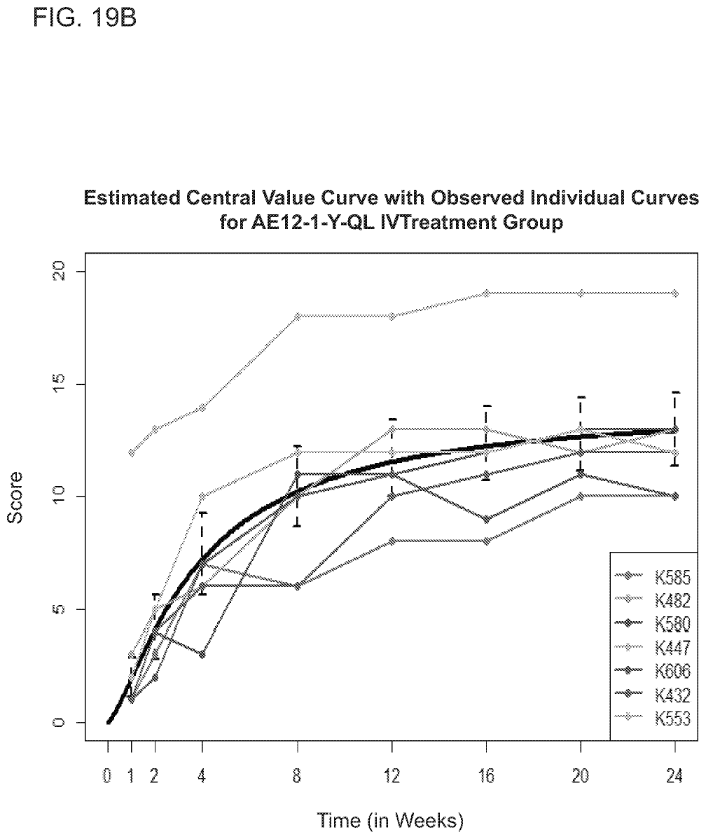

Observed neuromotor scores are represented graphically for each individual animal in FIGS. 19A and 19B. An estimated central value curve was generated.

In this severe, thoracic hemicompression model of SCI in non-human primates, chronic intravenous treatment with AE12-1-Y-QL demonstrated a beneficial effect over 6 months of recovery based upon blinded neuromotor scoring analysis. These clearly defined improvements in function with intravenous treatment with AE12-1-Y-QL were of comparable magnitude to that seen in the rat SCI model.

Continuous intrathecal infusion of AE12-1-Y-QL, however, showed a numerical improvement on neuromotor scores as compared to control, but the difference was small and not statistically significant. Unlike an intrathecal pilot study in uninjured non-human primates, which demonstrated predicted 24 hour+ exposure of AE12-1-Y-QL in CSF and serum, animals which underwent SCI had virtually no drug exposure 24 hours post SCI in serum or CSF, likely attributable to severe spinal edema and obstruction of CSF flow following SCI.

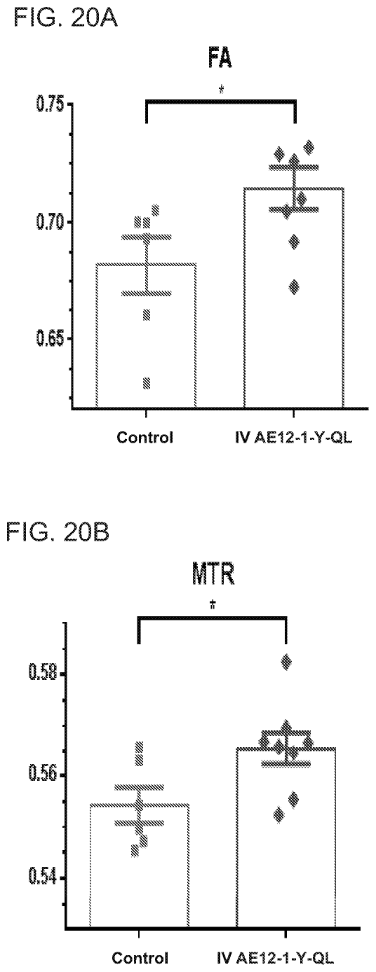

MRI analysis of the spinal cord further supports the efficacy of intravenous treatment with AE12-1-Y-QL. A T2 injury threshold (T2^{injury}) was defined as the T2-weighted image intensity at which the probability density of the injury distribution became higher than that of the normal white matter distribution. A histogram-based automated segmentation approach was used to define injured white matter in each slice of the thoracic T2-weighted MR scans. Regions of normal (extra-lesional) white matter and lesioned white matter were defined in 10 slices of a representative T2-weighted scan. Histograms of voxel intensity were constructed for each tissue and were fit with Gaussian functions. Based upon T2-defined regions of lesioned and extra-lesional white matter of 24-week old SCI thoracic spinal cord injuries, intravenous AE12-1-Y-QL demonstrated a greater preservation of tissue integrity in the extra-injury regions as compared to an IgG control group and an intrathetcal AE12-1-Y-QL group as quantified by magnetization transfer ratio (MTR) and fractional anisotropy (FA). Graphical representations of the data are in FIGS. 20A and 20B. These results suggest that treatment of SCI in non-human primates with intravenous administration of AE12-1-Y-QL, 75 min post injury, preserves tissue integrity of the extra-lesional spinal cord tissue to a greater extent than observed with IgG controls or by intrathecal administration of AE12-1-Y-QL.

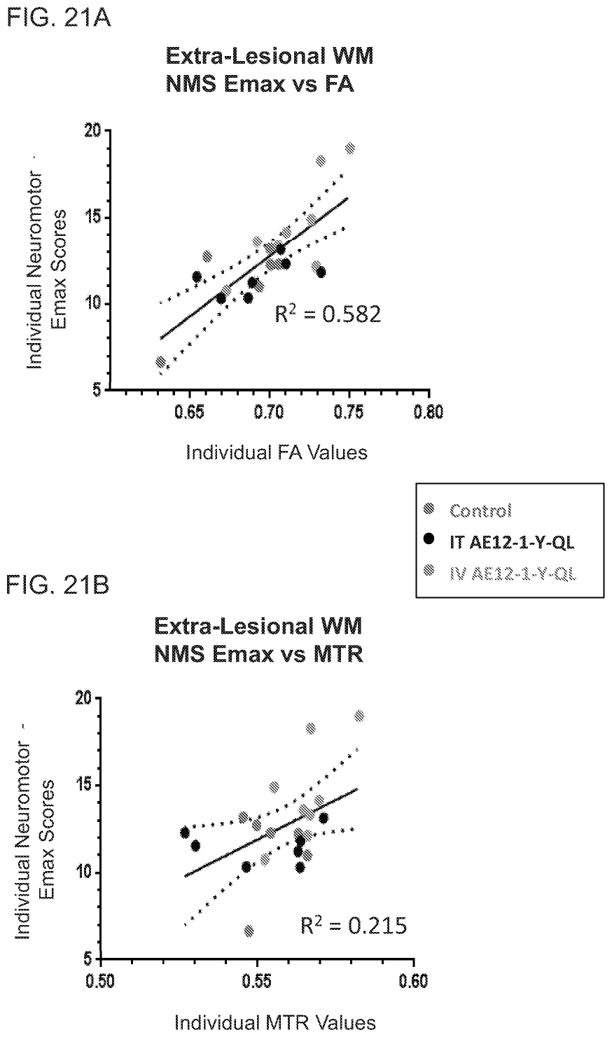

Moreover, neuromotor score values were positively correlated with extra-lesional white matter MTR and FA values, which reflect microstructural integrity. As shown in FIGS. 21A and 21B, the MTR and FA values generally increase with improved neuromotor function.

In summary, (i) at 24 weeks after intravenous treatment with AE12-1-Y-QL, there were significant increases in MTR and FA in the extra-lesional white matter compared to the control group, suggesting structural/functional improvement (or sparing of further secondary damage) by the treatment in extra-lesional white matter. In contrast, no significant changes were detected in any imaging endpoint between control and AE12-1-Y-QL-treated groups in the lesioned white matter; and (ii) there was a positive correlation between extra-lesional white matter FA or MTR with neuromotor scores Eₘₐₓ, suggesting that higher MTR and FA values, meaning improvement in extra-lesional white matter by intravenous treatment with AE12-1-Y-QL were associated with improved neuromotor function.

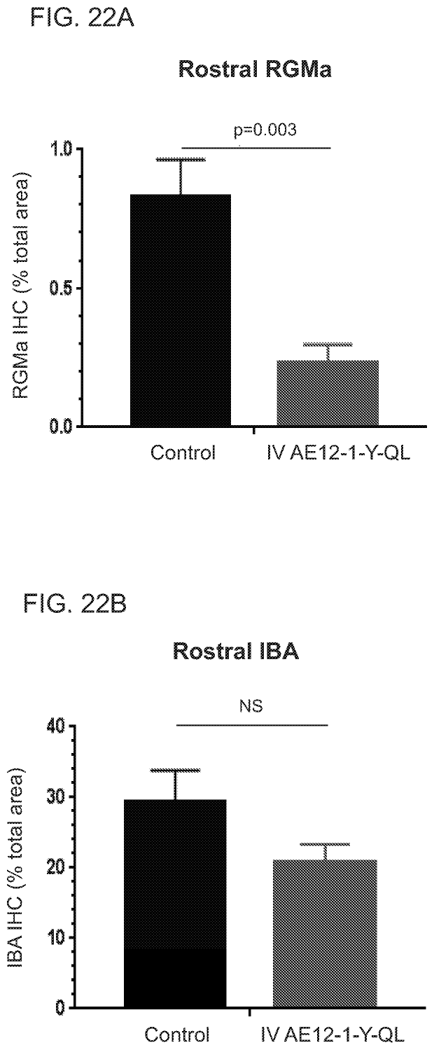

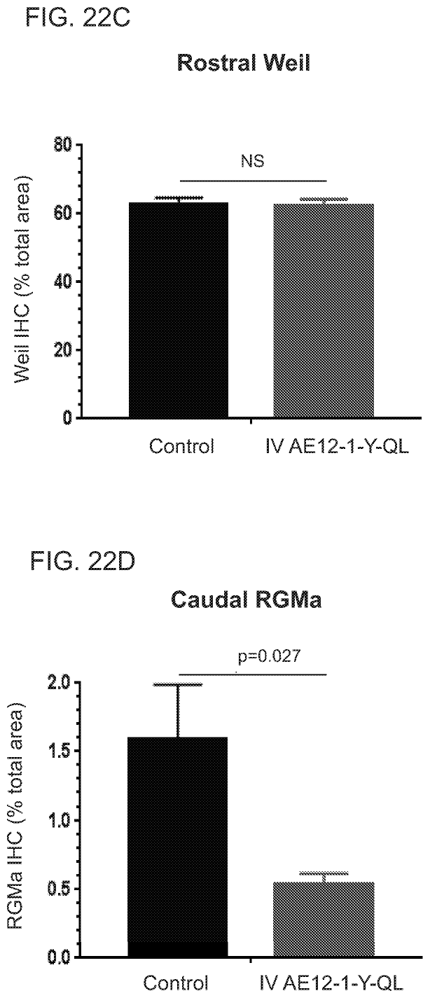

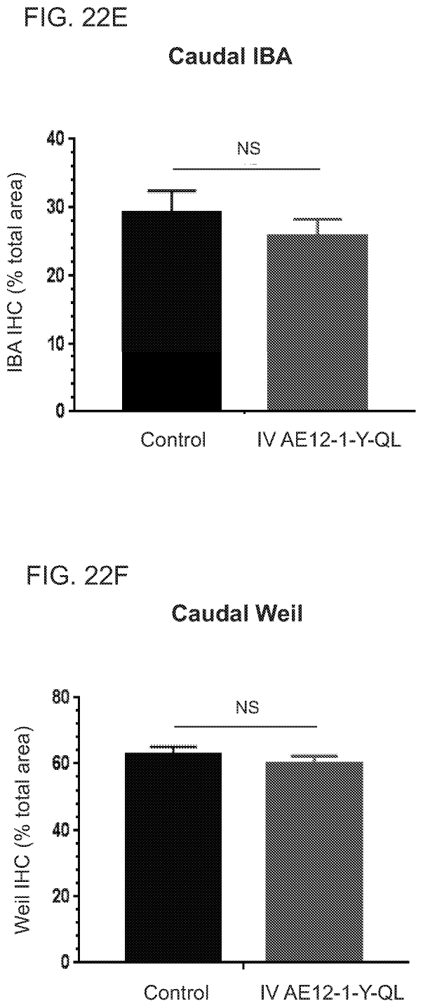

Histopathological analysis of spinal cord sections revealed significant differences in RGMa expression but not in a marker for activated microglia (ionized calcium binding adaptor molecule 1; IBA) or Weil staining of myelin. As shown in FIGS. 22A and 22D, rostral and caudal RGMA expression was significantly decreased after intravenous treatment with AE12-1-Y-QL.

### Example 3

As in Example 1, rats were pre-trained and then clip impact-compression SCI was made at spinal cord level T8 with a 20 g force for 1 min with a modified aneurysm clip according to published protocols. AE12-1-Y-QL (25 mg/kg) or an hIgG isotype control (25 mg/kg) was administered intravenously via tail vein acutely (within 5 min of the injury), or at 3 hr post-SCI, or 24 hr post-SCI, and then weekly for 6 weeks. There were five groups in the study: i) acute AE12-1-Y-QL (injected within 5 min of the injury), ii) acute hIgG (injected within 5 min of the injury), iii) 3 hr AE12-1-Y-QL, iv) 24 hr AE12-1-Y-QL, and v) 24 hr hIgG. All rats received weekly tail vein injections for 6 weeks post-SCI.

Methodology: Locomotor function was evaluated using the Basso, Beattie and Bresnahan (BBB) locomotor rating scale at 1 day post-SCI, and then weekly for 9 weeks post-SCI. To further elucidate motor function, gait analysis was performed using the CatWalk system (Noldus Information Technology, Wageningen, Netherlands). The following gait parameters were examined: a) regularity index which is a fractional measure of inter-paw coordination. In healthy normal animals, the regularity index is 100%, b) stride length which is the distance between successive placements of the same paw, c) swing speed which is the mean speed of paw during swing, and d) paw intensity which is a measure for the mean pressure exerted by the paw on the glass plate and depends on the degree of contact between the paw and the glass plate. Mechanical allodynia and thermal hyperalgesia were assessed as described above.

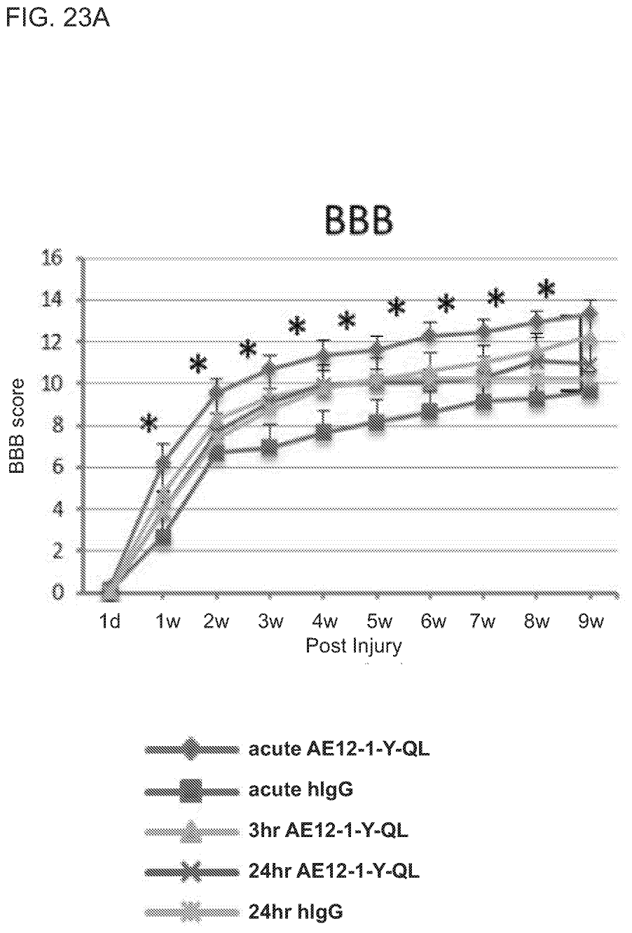

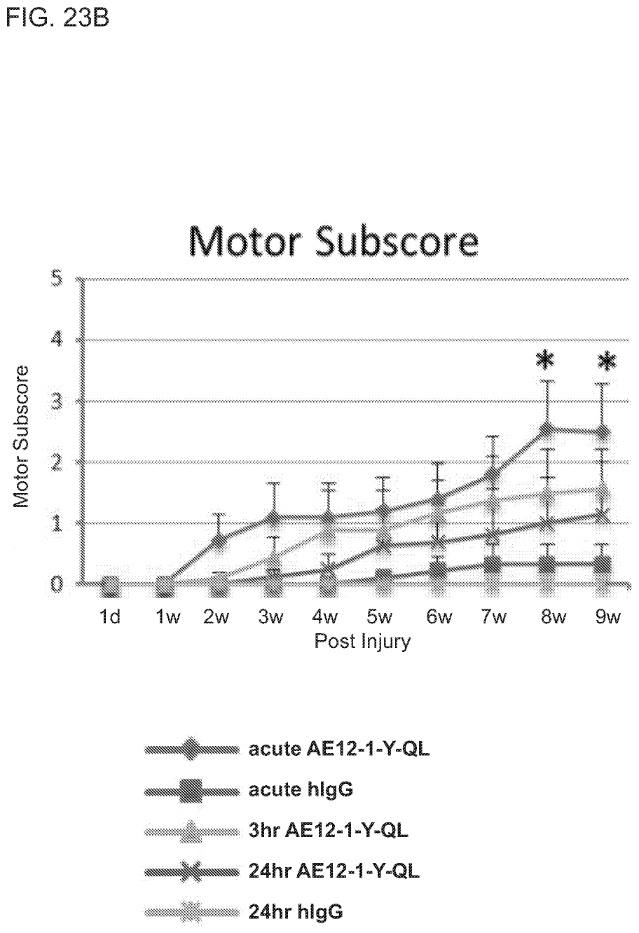

Results: Rats in the acute AE12-1-Y-QL group had significantly higher BBB scores relative to the control group (acute hIgG) throughout the time course post-SCI (FIG. 23A). There is a trend towards improved recovery in rats treated with AE12-1-Y-QL at 3 hr post injury. Also the scores in the acute and 3 hr AE12-1-Y-QL groups have not yet plateaued compared to the other groups. The acute AE12-1-Y-QL group shows a significantly higher motor subscore at 8 and 9 weeks post injury relative to control groups (FIG. 23B).

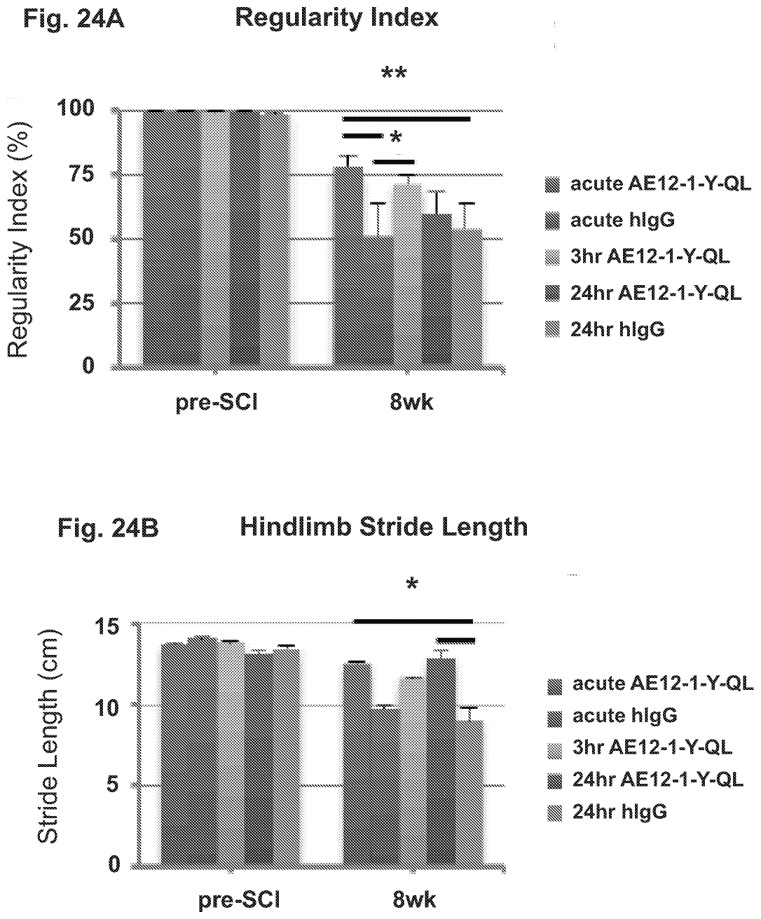

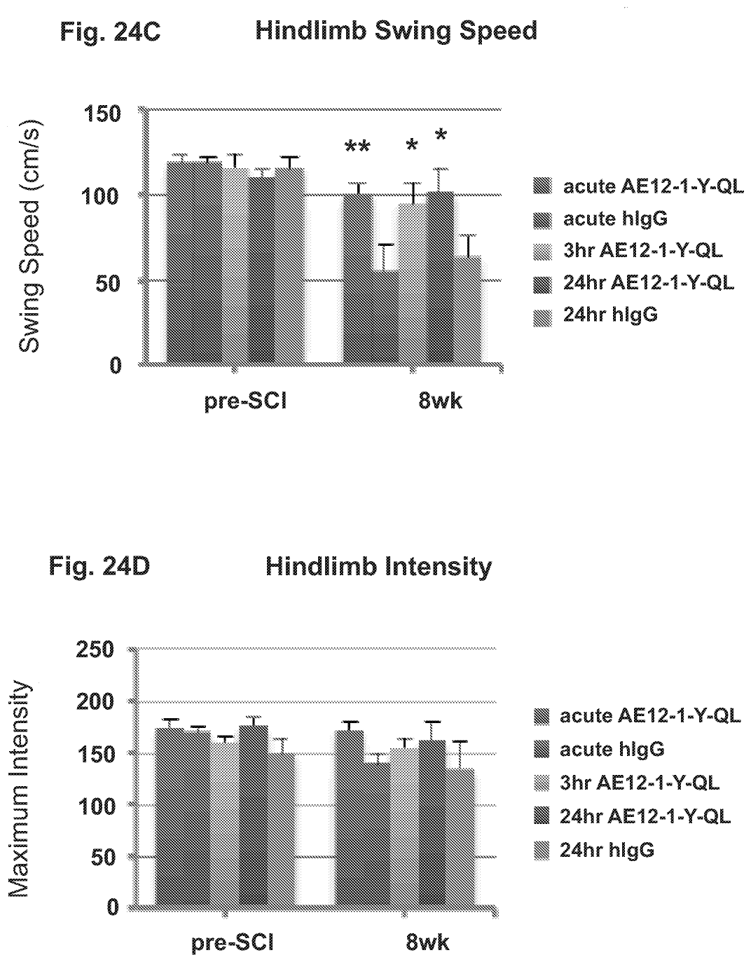

At 8 weeks post-SCI, the acute and 3 hr AE12-1-Y-QL groups had significantly higher regularity index scores relative to controls (FIG. 24A). Regularity index is a measure of inter-paw coordination. In healthy, fully coordinated animals the value is 100%.

At 8 weeks post-SCI, rats treated with AE12-1-Y-QL at all time window intervals showed a greater stride length; the difference was statistically significant in the acute and 24 hr AE12-1-Y-QL groups compared to 24 hr IgG controls (FIG. 24B). Stride length is the distance between successive placements of the same paw which decreases following SCI.

All treated groups showed a higher swing speed which was statistically significantly from both control groups (FIG. 24C). Swing speed is the speed of the paw during the swing phase which decreased after SCI.

At 8 weeks post-SCI, the acute AE12-1-Y-QL group showed a trend towards pre-SCI values for hindlimb intensity, which was not statistically significant from controls (FIG. 24D). Hindlimb intensity is a measure of the weight support of the hindllimbs.

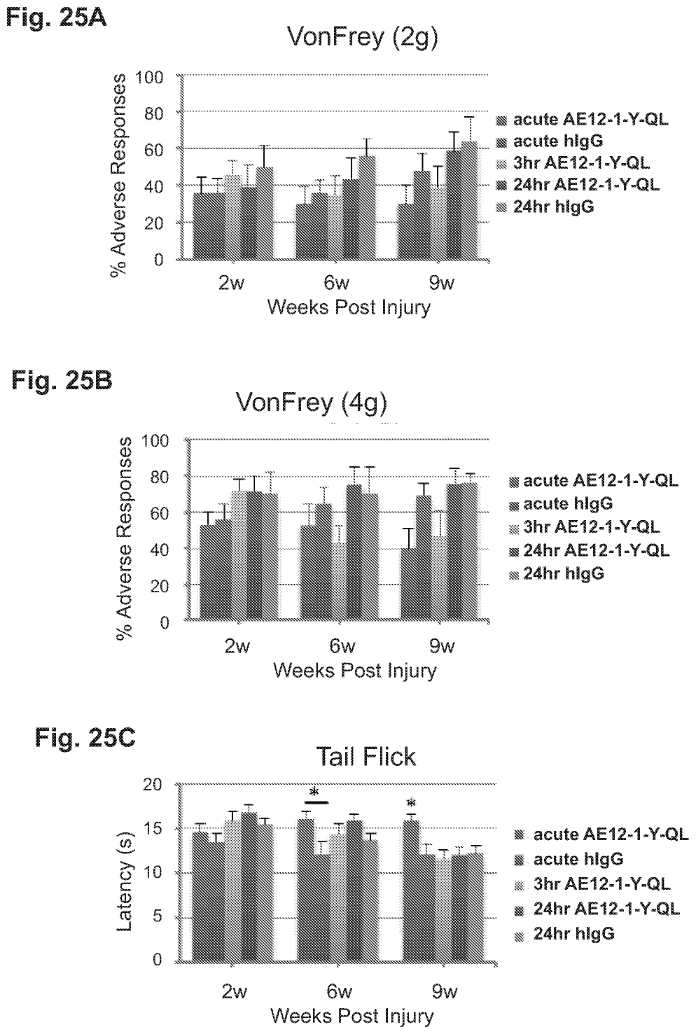

There were no significant differences between groups in at-level mechanical allodynia, although the acute and 3 hr AE12-1-Y-QL groups showed fewer adverse responses compared to controls at 9 weeks post-SCI with the 2 g filament and at 6 and 9 weeks post-SCI with the 4 g filament (FIGS. 25A and 25B).

At 6 weeks post-SCI and injections, the acute AE12-1-Y-QL group showed significantly increased latency to withdrawal to the heat stimulus compared to acute IgG controls (FIG. 25C). This effect was maintained at 9 weeks post SCI.

### 6. EXEMPLARY EMBODIMENTS

The following exemplary embodiments are provided:

1. A method of treating a spinal cord injury in a subject in need thereof, the method comprising administering a therapeutically effective amount of an antibody or antigen-binding fragment thereof that specifically binds Repulsive Guidance Molecule A (RGMa), wherein the antibody or antigen binding fragment comprises (a) a variable heavy chain comprising a complementarity determining region (VH CDR)-1 comprising an amino acid sequence of SEQ ID NO:1, a VH CDR-2 comprising an amino acid sequence of SEQ ID NO:2, and a VH CDR-3 comprising an amino acid sequence of SEQ ID NO:3; and (b) a variable light chain comprising a complementarity determining region (VL CDR)-1 comprising an amino acid sequence of SEQ ID NO:4, a VL CDR-2 comprising an amino acid sequence of SEQ ID NO:5, and a VL CDR-3 comprising an amino acid sequence selected from the group consisting of SEQ ID NO:6 and SEQ ID NO:7.

2. A method of promoting axonal regeneration, functional recovery, or both in a subject having a spinal cord injury, the method comprising administering a therapeutically effective amount of an antibody or antigen-binding fragment thereof that specifically binds Repulsive Guidance Molecule A (RGMa), wherein the antibody or antigen binding fragment comprises (a) a variable heavy chain comprising a complementarity determining region (VH CDR)-1 comprising an amino acid sequence of SEQ ID NO:1, a VH CDR-2 comprising an amino acid sequence of SEQ ID NO:2, and a VH CDR-3 comprising an amino acid sequence of SEQ ID NO:3; and (b) a variable light chain comprising a complementarity determining region (VL CDR)-1 comprising an amino acid sequence of SEQ ID NO:4, a VL CDR-2 comprising an amino acid sequence of SEQ ID NO:5, and a VL CDR-3 comprising an amino acid sequence selected from the group consisting of SEQ ID NO:6 and SEQ ID NO:7.

3. The method of embodiment 2, wherein the functional recovery is assessed by a neurobehavioral test.

4. The method of any one of embodiments 1-3, wherein the spinal cord injury is a compression or an impact injury.

5. The method of any one of embodiments 1-4, wherein the antibody is administered within 24 hours of the spinal cord injury.

6. A method of treating pain in a subject in need thereof, the method comprising administering a therapeutically effective amount of an antibody or antigen-binding fragment thereof that specifically binds Repulsive Guidance Molecule A (RGMa), wherein the antibody or antigen binding fragment comprises (a) a variable heavy chain comprising a complementarity determining region (VH CDR)-1 comprising an amino acid sequence of SEQ ID NO:1, a VH CDR-2 comprising an amino acid sequence of SEQ ID NO:2, and a VH CDR-3 comprising an amino acid sequence of SEQ ID NO:3; and (b) a variable light chain comprising a complementarity determining region (VL CDR)-1 comprising an amino acid sequence of SEQ ID NO:4, a VL CDR-2 comprising an amino acid sequence of SEQ ID NO:5, and a VL CDR-3 comprising an amino acid sequence selected from the group consisting of SEQ ID NO:6 and SEQ ID NO:7.

7. The method of embodiment 6, wherein the pain is neuropathic pain.

8. The method of embodiment 7, wherein the neuropathic pain arises from a spinal cord injury.

9. The method of embodiment 8, wherein the antibody is administered within 24 hours of the spinal cord injury.

10. The method of embodiment 7, wherein the neuropathic pain arises from chemotherapy.

11. The method of embodiment 7, wherein the neuropathic pain is postherpetic neuralgia.

12. The method of any one of embodiments 1-11, wherein the antibody or antigen-binding fragment thereof is administered systemically.

13. The method of any one of embodiments 1-12, wherein the antibody or antigen-binding fragment thereof is administered intravenously (IV).

14. The method of any one of embodiments 1-13, wherein the VL CDR-3 comprises an amino acid sequence of SEQ ID NO:6.

15. The method of any one of embodiments 1-13, wherein the VL CDR-3 comprises an amino acid sequence of SEQ ID NO:7.

16. The method of any one of embodiments 1-13, wherein the variable heavy chain comprises an amino acid sequence of SEQ ID NO: 8 and the variable light chain comprises an amino acid sequence of SEQ ID NO: 9.

17. The method of any one of embodiments 1-13, wherein the variable heavy chain comprises an amino acid sequence of SEQ ID NO: 8 and the variable light chain comprises an amino acid sequence of SEQ ID NO: 10.

18. The method of any one of embodiments 1-17, the antibody is selected from the group consisting of a human antibody, an immunoglobulin molecule, a disulfide linked Fv, a monoclonal antibody, an affinity matured antibody, a scFv, a chimeric antibody, a CDR-grafted antibody, a diabody, a humanized antibody, a multispecific antibody, a Fab, a dual specific antibody, a DVD, a Fab′, a bispecific antibody, a F(ab′)₂, and a Fv.

19. The method of embodiment 18, wherein the antibody is a human antibody.

20. The method of embodiment 18, wherein the antibody is a monoclonal antibody.

21. The method of any one of embodiments 1-13, wherein the antibody comprises a constant region comprising an amino acid sequence selected from the group consisting of SEQ ID NO: 11, SEQ ID NO: 12, SEQ ID NO: 13, and SEQ ID NO: 14.

22. The method of any one of embodiments 1-13, wherein the antibody comprises a heavy chain sequence of SEQ ID NO: 16 and a light chain sequence of SEQ ID NO: 15.

It is understood that the foregoing detailed description and accompanying examples and exemplary embodiments are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A method of treating a spinal cord injury in a subject in need thereof, the method comprising administering a therapeutically effective amount of a monoclonal anti-Repulsive Guidance Molecule A (RGMa) antibody, wherein the antibody comprises
a. a variable heavy chain comprising a complementarity determining region (VH CDR)-1 comprising an amino acid sequence of SEQ ID NO:1, a VH CDR-2 comprising an amino acid sequence of SEQ ID NO:2, and a VH CDR-3 comprising an amino acid sequence of SEQ ID NO:3; and
b. a variable light chain comprising a complementarity determining region (VL CDR)-1 comprising an amino acid sequence of SEQ ID NO:4, a VL CDR-2 comprising an amino acid sequence of SEQ ID NO:5, and a VL CDR-3 comprising an amino acid sequence selected from the group consisting of SEQ ID NO:6 and SEQ ID NO:7.

2. The method of claim 1, wherein the method comprises promoting axonal regeneration, functional recovery, or both following the spinal cord injury.

3. The method of claim 1, wherein the method comprises treating pain arising from the spinal cord injury.

4. The method of claim 3, wherein the pain is neuropathic pain.

5. The method of claim 1, wherein the spinal cord injury is a compression, a contusion, or an impact injury.

6. The method of claim 1, wherein the antibody is administered less than 8 hours post spinal cord injury.

7. The method of claim 1, wherein the monoclonal anti-RGMa antibody is administered systemically.

8. The method of claim 1, wherein the monoclonal anti-RGMa antibody is administered intravenously (IV).

9. The method of claim 1, wherein the VL CDR-3 comprises an amino acid sequence of SEQ ID NO:6.

10. The method of claim 1, wherein the VL CDR-3 comprises an amino acid sequence of SEQ ID NO:7.

11. The method of claim 1, wherein the variable heavy chain comprises an amino acid sequence of SEQ ID NO: 8 and the variable light chain comprises an amino acid sequence of SEQ ID NO: 9.

12. The method of claim 1, wherein the variable heavy chain comprises an amino acid sequence of SEQ ID NO: 8 and the variable light chain comprises an amino acid sequence of SEQ ID NO: 10.

13. The method of claim 1, wherein the monoclonal anti-RGMa antibody is a human antibody.

14. The method of claim 1, wherein the antibody comprises a constant region comprising an amino acid sequence selected from the group consisting of SEQ ID NO: 11, SEQ ID NO: 12, SEQ ID NO: 13, and SEQ ID NO: 14.

15. The method of claim 14, wherein the monoclonal anti-RGMa antibody comprises a constant region comprising an amino acid sequence consisting of SEQ ID NO: 14.

16. The method of claim 1, wherein the antibody comprises a heavy chain sequence of SEQ ID NO: 16 and a light chain sequence of SEQ ID NO: 15

17. The method of claim 1, wherein the monoclonal anti-RGMa antibody binds to an RGMa epitope located in the N-terminal region of RGMa, preferably to an RGMa epitope within the amino acids of SEQ ID NO:18, more preferably to an RGMa epitope within the amino acids of SEQ ID NO: 19.
